(19) United States
(12) Patent Application Publication
(10) Pub. No.: US 2023/0028064 A1
(43) Pub. Date: Jan. 26, 2023

(54) LIGHT-ABSORBING MATERIAL, RECORDING MEDIUM USING THE SAME, INFORMATION RECORDING METHOD AND INFORMATION READING METHOD

(71) Applicant: Panasonic Intellectual Property Management Co., Ltd., Osaka (JP)

(72) Inventors: MASAKO YOKOYAMA, Osaka (JP); NAOYA SAKATA, Hyogo (JP); KENJI TAGASHIRA, Osaka (JP); KOTA ANDO, Osaka (JP)

(21) Appl. No.: 17/929,301

(22) Filed: Sep. 1, 2022

Related U.S. Application Data
(63) Continuation of application No. PCT/JP2021/009974, filed on Mar. 12, 2021.

(30) Foreign Application Priority Data
Mar. 27, 2020 (JP) 2020-059077

(51) Int. Cl. G11B 7/004; C07C 63/66; C07C 69/76; C07C 43/215; C07C 22/08

(52) U.S. Cl. CPC G11B 7/004; C07C 63/66; C07C 69/76; C07C 43/215; C07C 22/08

(57) ABSTRACT

A light-absorbing material includes a compound represented by the formula (1) below. In the formula (1), L¹ to L³ are each independently represented by the formula (2) or (3) below:

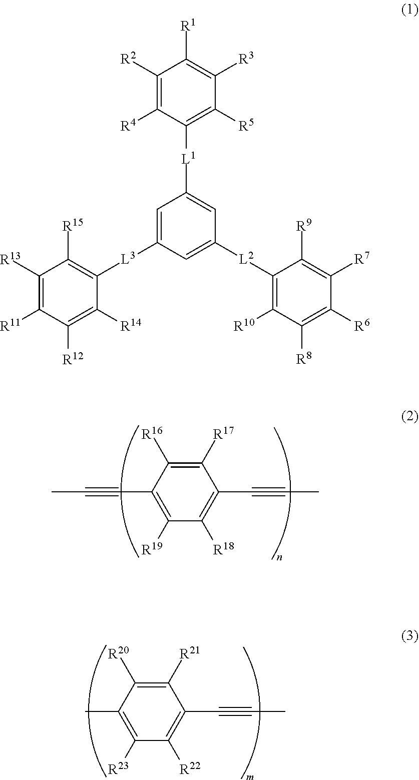

### BACKGROUND

### 1. Technical Field

The present disclosure relates to a light-absorbing material, a recording medium using the same, an information recording method, and an information reading method.

### 2. Description of the Related Art

Among optical materials such as light-absorbing materials, those materials having a non-linear optical effect are called non-linear optical materials. The non-linear optical effect means that when a substance is irradiated with intense light such as laser beam, an optical phenomenon proportional to the second or higher power of the electric field of the irradiation light occurs in the substance. Examples of the optical phenomena include absorption, reflection, scattering and light emission. Examples of the second-order non-linear optical effects proportional to the square of the electric field of irradiation light include second harmonic generation (SHG), Pockels effect and parametric effect. Examples of the third-order non-linear optical effects proportional to the cube of the electric field of irradiation light include two-photon absorption, multiphoton absorption, third harmonic generation (THG) and Kerr effect.

Non-linear optical materials have been an active topic in numerous studies. In particular, inorganic non-linear optical materials that can be easily prepared as single crystals have been developed. In recent years, the development of organic non-linear optical materials is expected. Compared to inorganic materials, organic materials not only offer a high degree of freedom in design but also have a high non-linear optical constant. Further, organic materials have a fast non-linear response. In the present specification, a non-linear optical material including an organic material is also written as an organic non-linear optical material.

### SUMMARY

In one general aspect, the techniques disclosed here feature a light-absorbing material containing a compound represented by the following formula (1):

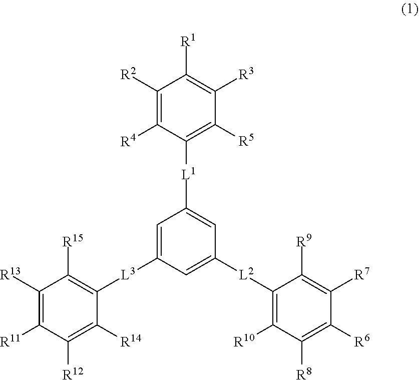

wherein R¹ to R¹⁵ each independently include at least one atom selected from the group consisting of H, C, N, O, F, P, S, Cl, I and Br, and L¹ to L³ are each independently represented by the following formula (2) or (3):

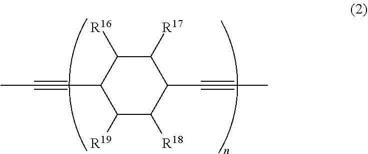

wherein R¹⁶ to R¹⁹ each independently include at least one atom selected from the group consisting of H, C, N, O, F, P, S, Cl, I and Br, and n is an integer of 1 to 3,

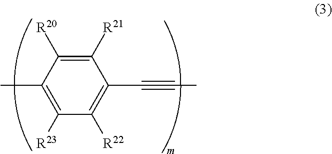

wherein R²⁰ to R²³ each independently include at least one atom selected from the group consisting of H, C, N, O, F, P, S, Cl, I and Br, and m is an integer of 1 to 3,

when the compound is represented by the formula (4) below, at least one selected from the group consisting of R¹, R⁶ and R¹¹ is a halogen atom, an alkyl group, an alkyl halide group, an unsaturated hydrocarbon group, a hydroxyl group, a carboxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group or a secondary amino group,

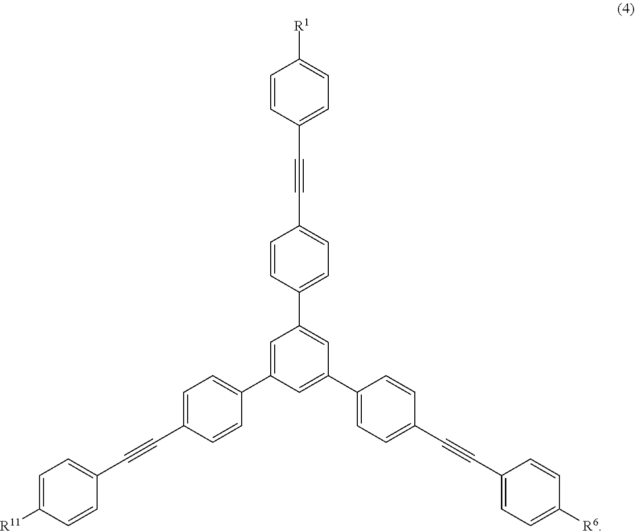

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flowchart regarding an information recording method using a recording medium including a light-absorbing material according to an embodiment of the present disclosure;

FIG. 1B is a flowchart regarding an information reading method using a recording medium including a light-absorbing material according to an embodiment of the present disclosure;

FIG. 2 is a graph illustrating a ¹H-NMR spectrum of compound (12)-1;

FIG. 3 is a graph illustrating a ¹H-NMR spectrum of compound (12)-7;

FIG. 4 is a graph illustrating a ¹H-NMR spectrum of compound (12)-9;

FIG. 5 is a graph illustrating a ¹H-NMR spectrum of compound (12)-10;

FIG. 6 is a graph illustrating a ¹H-NMR spectrum of compound (13)-7;

FIG. 7 is a graph illustrating a ¹H-NMR spectrum of compound (13)-10;

FIG. 8 is a graph illustrating a ¹H-NMR spectrum of compound (8)-5;

FIG. 9 is a graph illustrating a ¹H-NMR spectrum of compound (8)-7;

FIG. 10 is a graph illustrating a ¹H-NMR spectrum of compound (8)-9;

FIG. 11 is a graph illustrating a ¹H-NMR spectrum of compound (8)-10;

FIG. 12 is a graph illustrating a ¹H-NMR spectrum of compound (9)-7; and

FIG. 13 is a graph illustrating a ¹H-NMR spectrum of compound (10)-9.

### DETAILED DESCRIPTIONS

There is a demand for light-absorbing materials that exhibit highly non-linear two-photon absorption characteristics when irradiated with light having a wavelength in a short wavelength region.

The present disclosure provides a light-absorbing material that exhibits highly non-linear two-photon absorption characteristics when irradiated with light having a wavelength in a short wavelength region.

### Underlying Knowledge Forming Basis of the Present Disclosure

Two-photon absorption materials have attracted particular attention among other organic non-linear optical materials. Two-photon absorption means a phenomenon in which a compound is raised to an excited state by absorbing two photons almost at the same time. Two-photon absorption that occurs in a wavelength region where there is no single-photon absorption band is called non-resonant two-photon absorption. When a compound absorbs the first photon and further absorbs the second photon to be promoted to a higher-order excited state, the two-photon absorption is called resonant two-photon absorption. In resonant two-photon absorption, a compound absorbs two photons sequentially.

In non-resonant two-photon absorption, the amount of light absorbed by a compound is usually proportional to the square of the irradiation light intensity and is non-linear. The amount of light absorption may be used as an index of the efficiency of two-photon absorption. When the amount of light absorbed by a compound is non-linear, the absorption of light by the compound may be caused to occur exclusively near, for example, the laser focal point having a high electric field intensity. That is, in a sample including a two-photon absorption material, the compound may be excited at a desired location in a selective manner. Because of this very high spatial resolution, compounds having non-resonant two-photon absorption characteristics are studied for use in applications such as recording layers in three-dimensional optical memories and photocurable resin compositions for stereolithography. A two-photon absorption material that further has fluorescence characteristics may be used as a fluorescent dye material in, for example, a two-photon fluorescence microscope. The use of such a two-photon absorption material in a three-dimensional optical memory leads to a possibility that the ON/OFF state of a recording layer is read by a system based on a change in fluorescence from the two-photon absorption material. In the current optical memories, the ON/OFF state of a recording layer is read by a system based on a change in light reflectance and a change in light absorption rate of a light-absorbing material. When this system is applied to a three-dimensional optical memory, unfortunately, crosstalk may be caused by interference between the recording layer of interest and other recording layers.

A great number of organic two-photon absorption materials having a large two-photon absorption cross section have been proposed so far. The two-photon absorption cross section is an index showing the efficiency of two-photon absorption. The unit for the two-photon absorption cross section is GM (10⁻⁵⁰cm⁴·s·molecule⁻¹·photon⁻¹). So far, many compounds having as large a two-photon absorption cross section as more than 500 GM have been reported (for example, Harry L. Anderson et al., “Two-Photon Absorption and the Design of Two-Photon Dyes”, Angew. Chem. Int. Ed. 2009, Vol. 48, pp. 3244-3266). In most of the reports, the two-photon absorption cross section is measured using laser light with a wavelength longer than 600 nm. In particular, a near infrared ray having a wavelength longer than 750 nm is sometimes used as the laser light.

However, for industrial applications, two-photon absorption materials are required to have a large two-photon absorption cross section when irradiated with laser light having a shorter wavelength. In the field of, for example, three-dimensional optical memories, laser light having a shorter wavelength forms a finer focused spot and thus allows for enhancement in the recording density of the three-dimensional optical memories. In the stereolithographic field too, laser light having a shorter wavelength realizes higher resolution modeling. In particular, laser light with a central wavelength of 405 nm is used under the Blu-ray (registered trademark) disc standards. Thus, great contribution is made to the progress of industry by the development of a compound that has a large two-photon absorption cross section when irradiated with light in the same wavelength region as the above laser light.

Japanese Patent No. 5769151 and Japanese Patent No. 5659189 disclose compounds having a large two-photon absorption cross section with respect to light having a wavelength of about 405 nm. Japanese Patent No. 5821661 and Japanese Unexamined Patent Application Publication No. 2013-242939 disclose compounds that are contained in optical information recording media capable of writing information in a shortened time when irradiated with laser light having a wavelength of about 405 nm.

Japanese Patent No. 5769151 describes a benzene derivative that has a structure formed by extension of a π-electron conjugated system. While this benzene derivative attains an increased two-photon absorption cross section due to the extension of the π-electron conjugated system, its single-photon absorption peak shifts toward a long wavelength region. As a result, part of the wavelengths giving rise to a single-photon absorption peak overlaps with the wavelength of the excitation light. For example, the wavelength of the excitation light is 405 nm defined by the Blu-ray (registered trademark) standards. The occurrence of single-photon absorption of the excitation light lowers the non-linearity of two-photon absorption in the compound. Decreased non-linearity of two-photon absorption causes a serious problem in, for example, multilayering of recording layers in a three-dimensional optical memory. Japanese Patent No. 5659189 describes a benzophenone derivative having a highly planar π-electron conjugated system. In this benzophenone derivative, however, the quantum yield of intersystem crossing is almost 100%. The benzophenone derivative rapidly moves from the singlet excited state to the triplet excited state and emits almost no fluorescence.

The present inventors have newly found that a compound of the formula (1) described later has excellent two-photon absorption characteristics and low single-photon absorption characteristics when irradiated with light having a wavelength in a short wavelength region, and have developed a light-absorbing material of the present disclosure. In the present specification, the short wavelength region means a wavelength region including 405 nm, for example, a wavelength region of greater than or equal to 390 nm and less than or equal to 420 nm. In particular, the compound represented by the formula (1) has a large two-photon absorption cross section with respect to light having a wavelength of about 405 nm. Further, this compound has a small single-photon absorbance with respect to light having a wavelength of about 405 nm. In other words, the compound has highly non-linear two-photon absorption characteristics when irradiated with light having a wavelength of about 405 nm. Furthermore, the compound also tends to have a high fluorescence quantum yield.

(Summary of One Aspect of the Present Disclosure)

A light-absorbing material according to the first aspect of the present disclosure contains:

a compound represented by the following formula (1):

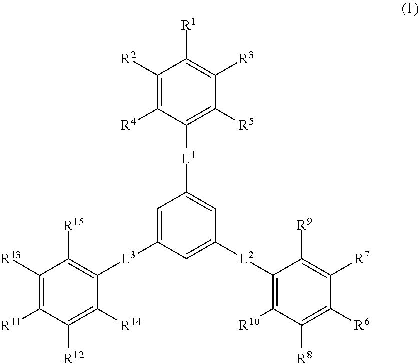

wherein R¹ to R¹⁵ each independently include at least one atom selected from the group consisting of H, C, N, O, F, P, S, Cl, I and Br, and L² to L³ are each independently represented by the following formula (2) or (3):

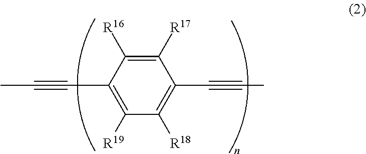

wherein R¹⁶ to R¹⁹ each independently include at least one atom selected from the group consisting of H, C, N, O, F, P, S, Cl, I and Br, and n is an integer of 1 to 3,

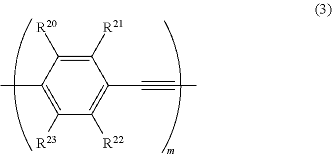

wherein R²⁰ to R²³ each independently include at least one atom selected from the group consisting of H, C, N, O, F, P, S, Cl, I and Br, and m is an integer of 1 to 3,

when the compound is represented by the formula (4) below, at least one selected from the group consisting of R¹, R⁶ and R¹¹ is a halogen atom, an alkyl group, an alkyl halide group, an unsaturated hydrocarbon group, a hydroxyl group, a carboxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group or a secondary amino group.

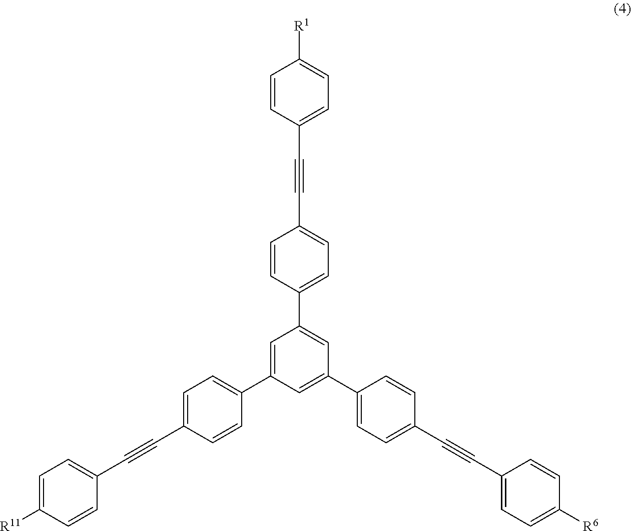

According to the first aspect, the light-absorbing material has excellent two-photon absorption characteristics and low single-photon absorption characteristics when irradiated with light having a wavelength in the short wavelength region. That is, the light-absorbing material has highly non-linear two-photon absorption characteristics with respect to light having a wavelength in the short wavelength region. The light-absorbing material also tends to have a high fluorescence quantum yield.

In the second aspect of the present disclosure, for example, the light-absorbing material according to the first aspect may be such that when the compound is represented by the formula (5) below, at least one selected from the group consisting of R¹, R⁶ and R¹¹ is a halogen atom, an alkyl group having two or more carbon atoms, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a primary amino group or a secondary amino group.

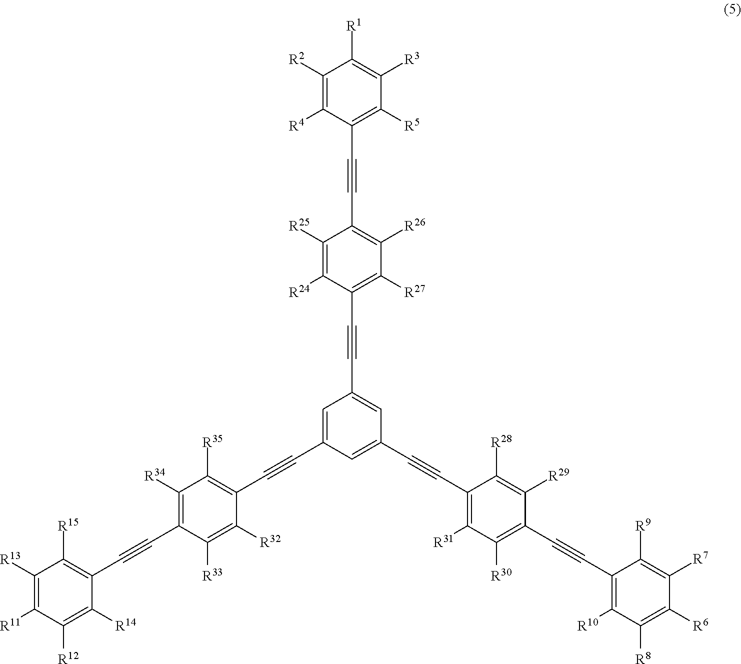

In the third aspect of the present disclosure, for example, the light-absorbing material according to the first aspect may be such that when the compound is represented by the formula (17) below, at least one selected from the group consisting of R¹, R⁶ and R¹¹ is a halogen atom, an alkyl group having two or more carbon atoms, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a silyl group, a primary amino group or a secondary amino group.

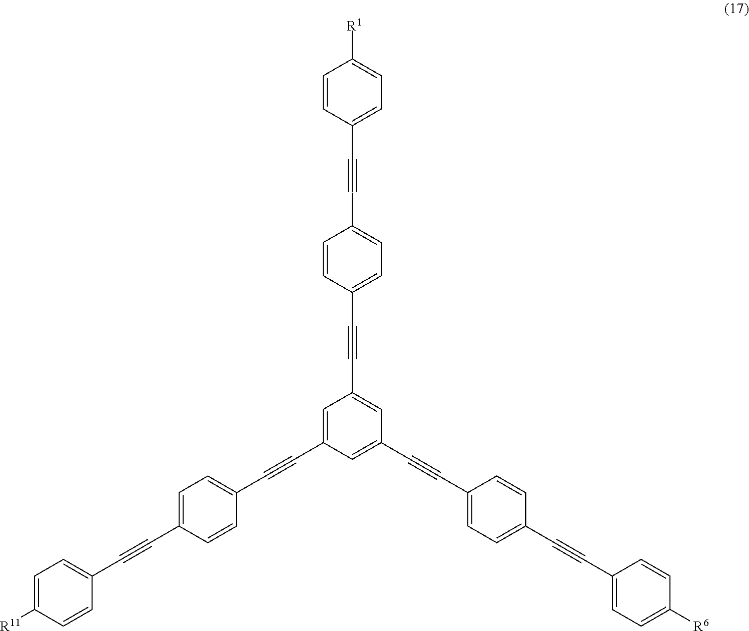

In the fourth aspect of the present disclosure, for example, the light-absorbing material according to the first aspect may be such that L¹ to L³ in the compound are each represented by the formula (2).

In the fifth aspect of the present disclosure, for example, the light-absorbing material according to the fourth aspect may be such that the compound is represented by the following formula (5):

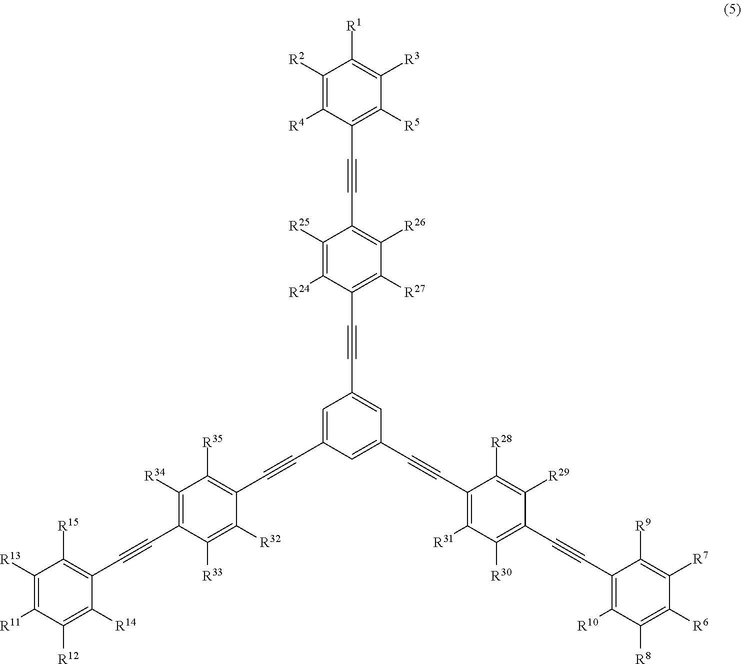

wherein R²⁴ to R³⁵ each independently include at least one atom selected from the group consisting of H, C, N, O, F, P, S, Cl, I and Br.

In the sixth aspect of the present disclosure, for example, the light-absorbing material according to the first aspect may be such that L¹ to L³ in the compound are each represented by the formula (3).

In the seventh aspect of the present disclosure, for example, the light-absorbing material according to the sixth aspect may be such that the compound is represented by the following formula (6):

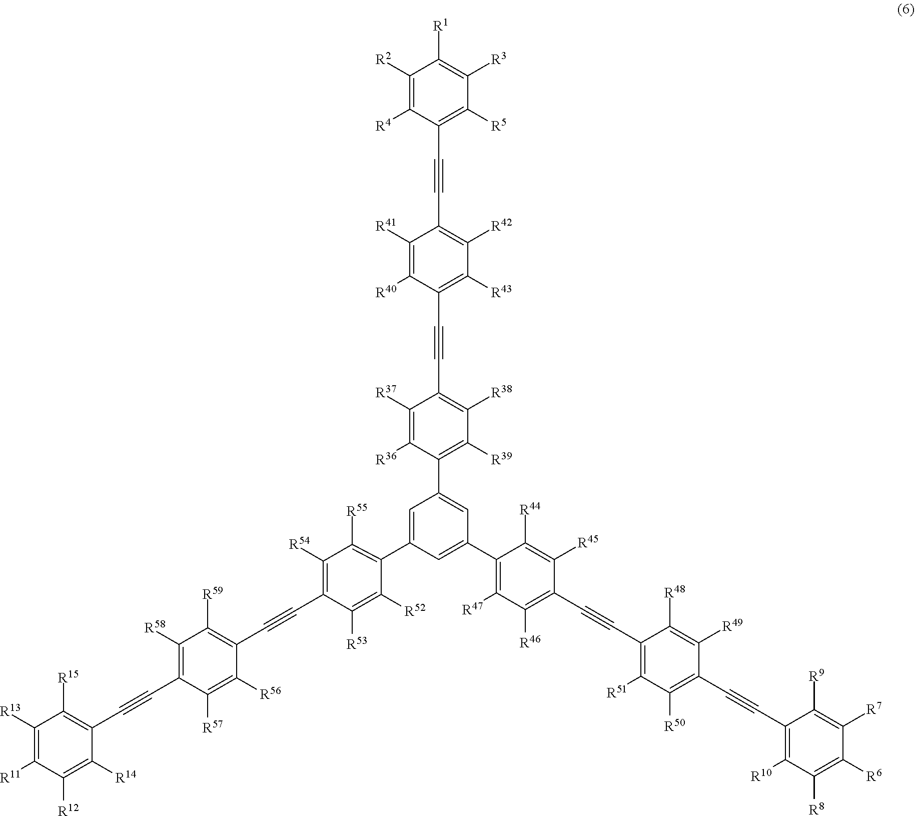

wherein R³⁶ to R⁵⁹ each independently include at least one atom selected from the group consisting of H, C, N, O, F, P, S, Cl, I and Br.

In the eighth aspect of the present disclosure, for example, the light-absorbing material according to the sixth aspect may be such that the compound is represented by the following formula (7):

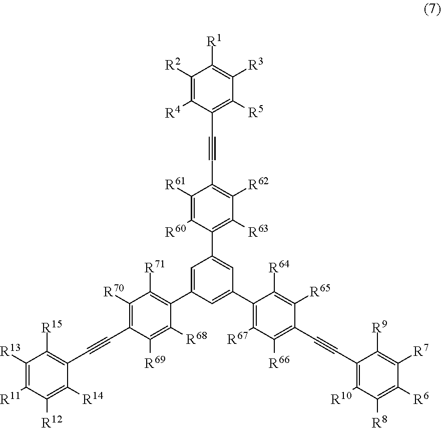

wherein R⁶⁰ to R⁷¹ each independently include at least one atom selected from the group consisting of H, C, N, O, F, P, S, Cl, I and Br.

In the ninth aspect of the present disclosure, for example, the light-absorbing material according to any one of the first to the eighth aspect may be such that R¹ to R¹⁵ are each independently a hydrogen atom, a halogen atom, an alkyl group, an alkyl halide group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group or a nitro group.

According to the second to ninth aspects, the light-absorbing materials have highly non-linear two-photon absorption characteristics with respect to light having a wavelength in the short wavelength region.

In the tenth aspect of the present disclosure, for example, the light-absorbing material according to any one of the first to the ninth aspect may be such that at least one selected from the group consisting of R¹ to R³, R⁶ to R⁸ and R¹¹ to R¹³ may be an electron-donating group or an electron-withdrawing group.

In the eleventh aspect of the present disclosure, for example, the light-absorbing material according to the tenth aspect may be such that the electron-withdrawing group is a carboxyl group or an alkoxycarbonyl group.

In the twelfth aspect of the present disclosure, for example, the light-absorbing material according to the tenth or the eleventh aspect may be such that the electron-withdrawing group is —COO(CH₂)₃CH₃ or —COO(CH₂)₇CH₃.

According to the tenth to twelfth aspects, the light-absorbing materials have higher two-photon absorption characteristics with respect to light having a wavelength in the short wavelength region.

A light-absorbing material according to the thirteenth aspect of the present disclosure is:

a light-absorbing material used in a device utilizing light having a wavelength of greater than or equal to 390 nm and less than or equal to 420 nm, the light-absorbing material containing a compound represented by the following formula (1):

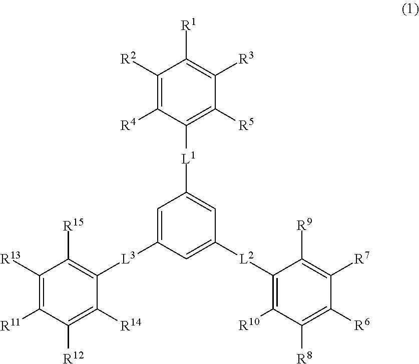

wherein R¹ to R¹⁵ each independently include at least one atom selected from the group consisting of H, C, N, O, F, P, S, Cl, I and Br, and L¹ to L³ are each independently represented by the following formula (2) or (3):

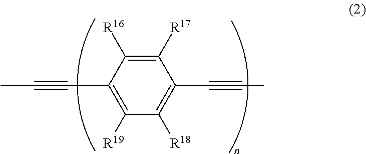

wherein R¹⁶ to R¹⁹ each independently include at least one atom selected from the group consisting of H, C, N, O, F, P, S, Cl, I and Br, and n is an integer of 1 to 3,

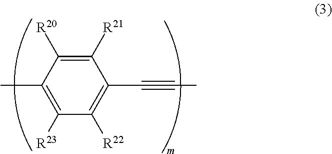

wherein R²⁰ to R²³ each independently include at least one atom selected from the group consisting of H, C, N, O, F, P, S, Cl, I and Br, and m is an integer of 1 to 3.

According to the thirteenth aspect, the light-absorbing material has excellent two-photon absorption characteristics and low single-photon absorption characteristics when irradiated with light having a wavelength in the short wavelength region. That is, the light-absorbing material has highly non-linear two-photon absorption characteristics with respect to light having a wavelength in the short wavelength region. The light-absorbing material also tends to have a high fluorescence quantum yield.

A recording medium according to the fourteenth aspect of the present disclosure includes:

a recording film containing a light-absorbing material containing a compound represented by the following formula (1):

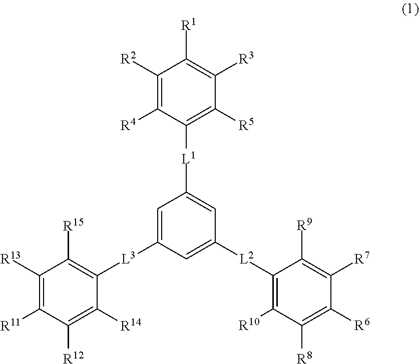

wherein R¹ to R¹⁵ each independently include at least one atom selected from the group consisting of H, C, N, O, F, P, S, Cl, I and Br, and L¹ to L³ are each independently represented by the following formula (2) or (3):

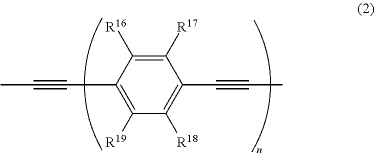

wherein R¹⁶ to R¹⁹ each independently include at least one atom selected from the group consisting of H, C, N, O, F, P, S, Cl, I and Br, and n is an integer of 1 to 3,

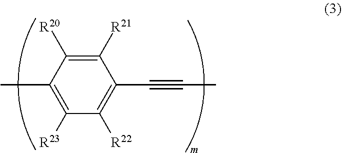

wherein R²⁰ to R²³ each independently include at least one atom selected from the group consisting of H, C, N, O, F, P, S, Cl, I and Br, and m is an integer of 1 to 3.

According to the fourteenth aspect, the light-absorbing material has excellent two-photon absorption characteristics and low single-photon absorption characteristics when irradiated with light having a wavelength in the short wavelength region. That is, the light-absorbing material has highly non-linear two-photon absorption characteristics with respect to light having a wavelength in the short wavelength region. The light-absorbing material also tends to have a high fluorescence quantum yield. The recording medium that includes a recording film including this light-absorbing material is suited for recording information in or reading information out of the recording medium.

An information recording method according to the fifteenth aspect of the present disclosure includes:

providing a light source that emits light having a wavelength of greater than or equal to 390 nm and less than or equal to 420 nm; and

focusing the light from the light source with a lens and applying the light to a recording region in a recording medium including a recording film containing a light-absorbing material containing a compound represented by the following formula (1):

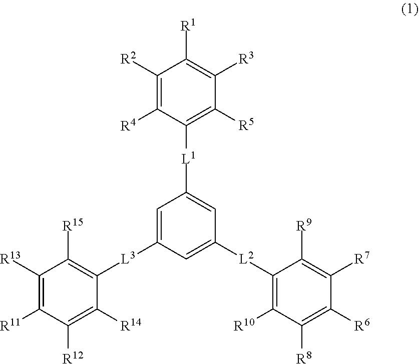

wherein R¹ to R¹⁵ each independently include at least one atom selected from the group consisting of H, C, N, O, F, P, S, Cl, I and Br, and L¹ to L³ are each independently represented by the following formula (2) or (3):

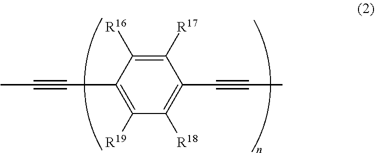

wherein R¹⁶ to R¹⁹ each independently include at least one atom selected from the group consisting of H, C, N, O, F, P, S, Cl, I and Br, and n is an integer of 1 to 3,

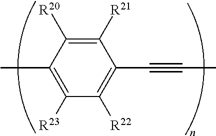

wherein R²⁰ to R²³ each independently include at least one atom selected from the group consisting of H, C, N, O, F, P, S, Cl, I and Br, and m is an integer of 1 to 3.

According to the fifteenth aspect, the light-absorbing material has excellent two-photon absorption characteristics and low single-photon absorption characteristics when irradiated with light having a wavelength in the short wavelength region. That is, the light-absorbing material has highly non-linear two-photon absorption characteristics with respect to light having a wavelength in the short wavelength region. By virtue of the use of the recording medium including such a light-absorbing material, the information recording method can record information with a high recording density.

An information reading method according to the sixteenth aspect of the present disclosure is a method for reading information recorded by, for example, the information recording method according to the fifteenth aspect,
the information reading method including:
   applying light to the recording region in the recording medium and measuring an optical characteristic of the recording region; and
   determining whether or not information is recorded in the recording region based on the optical characteristic.

In the seventeenth aspect of the present disclosure, for example, the information reading method according to the sixteenth aspect may be such that the optical characteristic is the intensity of fluorescence emitted from the recording region.

According to the sixteenth or seventeenth aspect, information may be read while suppressing the occurrence of crosstalk caused by interference between the recording region and other recording regions.

Hereinbelow, embodiments of the present disclosure will be described with reference to the drawings. The present disclosure is not limited to the embodiments discussed below.

A light-absorbing material of the present embodiment includes compound A represented by the following formula (1):

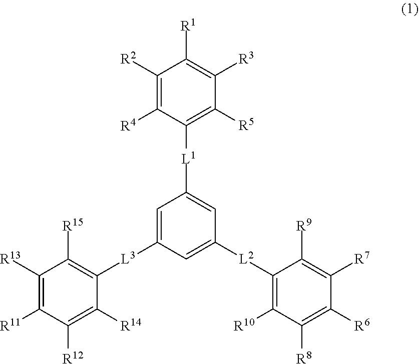

In the formula (1), R¹ to R¹⁵ each independently include at least one atom selected from the group consisting of H, C, N, O, F, P, S, Cl, I and Br. R¹ to R¹⁵ may be each independently a hydrogen atom, a halogen atom, an alkyl group, an alkyl halide group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group or a nitro group. R¹ to R¹⁵ may be each independently a hydrogen atom, a halogen atom, an alkyl group, an alkyl halide group, an unsaturated hydrocarbon group, a hydroxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group or a nitro group.

Examples of the halogen atoms include F, Cl, Br and I. In the present specification, a halogen atom may be referred to as a halogen group.

The number of carbon atoms in the alkyl group is not particularly limited, and is, for example, greater than or equal to 1 and less than or equal to 20. From the point of view of easy synthesis of compound A, the number of carbon atoms in the alkyl group may be greater than or equal to 1 and less than or equal to 10, or greater than or equal to 1 and less than or equal to 5. The solubility of compound A in a solvent or a resin composition may be controlled by controlling the number of carbon atoms in the alkyl group. The alkyl group may be linear, branched or cyclic. The alkyl group may be substituted with a group containing at least one atom selected from the group consisting of N, O, P and S, in place of at least one hydrogen atom. Examples of the alkyl groups include methyl group, ethyl group, propyl group, butyl group, 2-methylbutyl group, pentyl group, hexyl group, 2,3-dimethylhexyl group, heptyl group, octyl group, nonyl group, decyl group, undecyl group, dodecyl group, tridecyl group, tetradecyl group, pentadecyl group, hexadecyl group, heptadecyl group, octadecyl group, nonadecyl group, eicosyl group, 2-methoxybutyl group and 6-methoxyhexyl group.

The alkyl halide group means an alkyl group substituted with a halogen atom in place of at least one hydrogen atom. In the alkyl halide group, all the hydrogen atoms contained in the alkyl group may be substituted by halogen atoms. Examples of the alkyl groups include those described above. Specific examples of the alkyl halide groups include —CF₃.

The unsaturated hydrocarbon group includes an unsaturated bond such as a carbon-carbon double bond or a carbon-carbon triple bond. The number of unsaturated bonds contained in the unsaturated hydrocarbon group is, for example, greater than or equal to 1 and less than or equal to 5. The number of carbon atoms in the unsaturated hydrocarbon group is not particularly limited, and may be, for example, greater than or equal to 2 and less than or equal to 20, greater than or equal to 2 and less than or equal to 10, or greater than or equal to 2 and less than or equal to 5. The unsaturated hydrocarbon group may be linear, branched or cyclic. The unsaturated hydrocarbon group may be substituted with a group containing at least one atom selected from the group consisting of N, O, P and S, in place of at least one hydrogen atom. Examples of the unsaturated hydrocarbon groups include vinyl group and ethynyl group.

The hydroxyl group is represented by —OH. The carboxyl group is represented by —COOH. The alkoxycarbonyl group is represented by —COORₐ. The acyl group is represented by —COR_{b}. The amide group is represented by —CONR_{c}R_{d}. The nitrile group is represented by —CN. The alkoxy group is represented by —ORₑ. The acyloxy group is represented by —OCOR_{f}. The thiol group is represented by —SH. The alkylthio group is represented by —SR_{g}. The sulfonic acid group is represented by —SO₃H. The acylthio group is represented by —SCORₕ. The alkylsulfonyl group is represented by —SO₂Rᵢ. The sulfonamide group is represented by —SO₂NRⱼRₖ. The primary amino group is represented by —NH₂. The secondary amino group is represented ny —NHR₁. The tertiary amino group is represented by —NRₘRₙ. The nitro group is represented by —NO₂. Rₐ to Rₙ are each independently an alkyl group. Examples of the alkyl groups include those described hereinabove. R_{c} and R_{d} in the amide group, and Rⱼ and Rₖ in the sulfonamide group may be each independently a hydrogen atom.

Specific examples of the alkoxycarbonyl groups include —COOCH₃, —COO(CH₂)₃CH₃ and —COO(CH₂)₇CH₃. Specific examples of the acyl groups include —COCH₃. Specific examples of the amide groups include —CONH₂. Specific examples of the alkoxy groups include methoxy group, ethoxy group, 2-methoxyethoxy group, butoxy group, 2-methylbutoxy group, 2-methoxybutoxy group, 4-ethylthiobutoxy group, pentyloxy group, hexyloxy group, heptyloxy group, octyloxy group, nonyloxy group, decyloxy group, undecyloxy group, dodecyloxy group, tridecyloxy group, tetradecyloxy group, pentadecyloxy group, hexadecyloxy group, heptadecyloxy group, octadecyloxy group, nonadecyloxy group and eicosyloxy group. Specific examples of the acyloxy groups include —OCOCH₃. Specific examples of the acylthio groups include —SCOCH₃. Specific examples of the alkylsulfonyl groups include —SO₂CH₃. Specific examples of the sulfonamide groups include —SO₂NH₂. Specific examples of the tertiary amino groups include —N(CH₃)₂.

At least one selected from the group consisting of R¹ to R³, R⁶ to R⁸ and R¹¹ to R¹³ may be an electron-donating group or an electron-withdrawing group. The greater the electron-donating properties or the electron-withdrawing properties of any of R¹ to R³, R⁶ to R⁸ and R¹¹ to R¹³, the more unevenly the electrons are distributed in the compound A. When the electrons in the compound A are unevenly distributed to a great extent, the electrons tend to move significantly in the compound A when the compound A is excited. Such compound A tends to have higher two-photon absorption characteristics. In other words, the compound A tends to have a large two-photon absorption cross section when at least one selected from the group consisting of R¹ to R³, R⁶ to R⁸ and R¹¹ to R¹³ is an electron-donating group or an electron-withdrawing group.

The electron-withdrawing group means, for example, a substituent having a positive value of the substituent constant σₚ in the Hammett equation. Examples of the electron-withdrawing groups include halogen atoms, carboxyl groups, nitro groups, thiol groups, sulfonic acid groups, acyloxy groups, alkylthio groups, alkylsulfonyl groups, sulfonamide groups, acyl groups, acylthio groups, alkoxycarbonyl groups and alkyl halide groups. The electron-withdrawing group may be a carboxyl group or an alkoxycarbonyl group, or may be —COO(CH₂)₃CH₃ or —COO(CH₂)₇CH₃.

The electron-donating group means, for example, a substituent having a negative value of the above constant σₚ. Examples of the electron-donating groups include alkyl groups, alkoxy groups, hydroxyl groups and amino groups.

R⁴, R⁵, R⁹, R¹⁰, R¹⁴ and R¹⁵ may each have a small volume. In this case, steric hindrance is unlikely to occur in R⁴, R⁵, R⁹, R¹⁰, R¹⁴ and R¹⁵, and consequently the planarity of the π-electron conjugated system in the compound A tends to be enhanced. When the π-electron conjugated system in the compound A has high planarity, the compound A tends to have a large two-photon absorption cross section. R⁴, R⁵, R⁹, R¹⁰, R¹⁴ and R¹⁵ may be each a hydrogen atom.

In the formula (1), L¹ to L³ are each independently represented by the following formula (2) or (3).

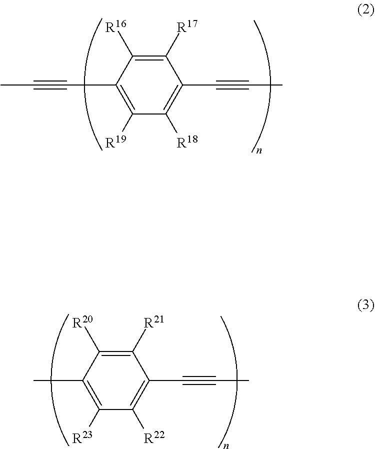

In the formula (2), R¹⁶ to R¹⁹ each independently include at least one atom selected from the group consisting of H, C, N, O, F, P, S, Cl, I and Br. R¹⁶ to R¹⁹ may be each independently a hydrogen atom or any of the substituents described hereinabove with respect to R¹ to R¹⁵. R¹⁶ to R¹⁹ may each have a small volume. In this case, steric hindrance is unlikely to occur in R¹⁶ to R¹⁹, and consequently the planarity of the π-electron conjugated system in the compound A is enhanced and the compound A tends to have a large two-photon absorption cross section. R¹⁶ to R¹⁹ may be each a hydrogen atom. In the formula (2), n is an integer of 1 to 3. With increasing value of n, the π-electron conjugated system is more extended and the compound A tends to have a larger two-photon absorption cross section. In consideration of the solubility of the compound A, n may be 1 or 2, or may be 1.

In the formula (3), R²⁰ to R²³ each independently include at least one atom selected from the group consisting of H, C, N, O, F, P, S, Cl, I and Br. R²⁰ to R²³ may be each independently a hydrogen atom or any of the substituents described hereinabove with respect to R¹ to R¹⁵. R²⁰ to R²³ may each have a small volume. In this case, steric hindrance is unlikely to occur in R²⁰ to R²³, and consequently the planarity of the π-electron conjugated system in the compound A is enhanced and the compound A tends to have a large two-photon absorption cross section. R²⁰ to R²³ may be each a hydrogen atom. In the formula (3), m is an integer of 1 to 3. With increasing value of m, the π-electron conjugated system is more extended and the compound A tends to have a larger two-photon absorption cross section. In consideration of the solubility of the compound A, m may be 1 or 2.

L¹ to L³ may be the same as or different from one another. For example, L¹ to L³ may be each represented by the formula (2). The compound A may be, for example, compound B represented by the following formula (5):

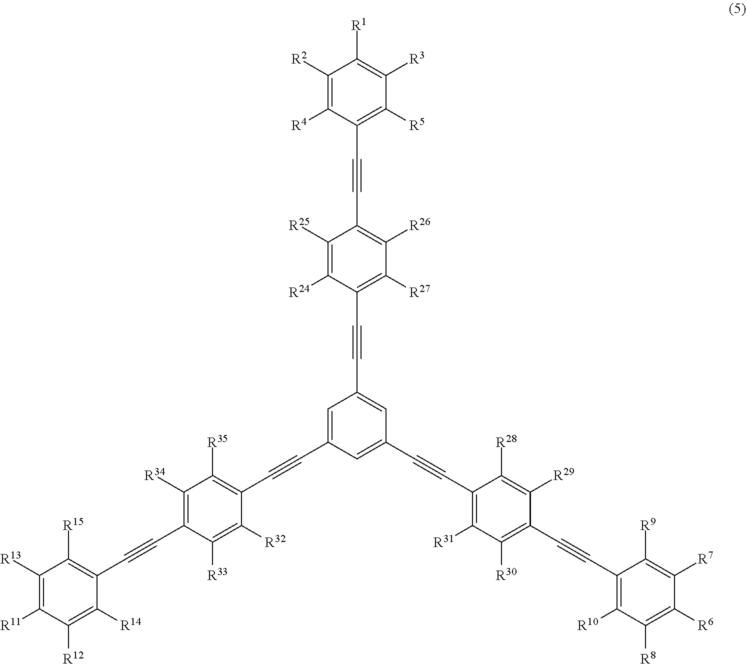

In the formula (5), R²⁴ to R³⁵ each independently include at least one atom selected from the group consisting of H, C, N, O, F, P, S, Cl, I and Br. Each of R²⁴ to R³⁵ corresponds to any of R¹⁶ to R¹⁹ described hereinabove.

Specific examples of the compounds B include compounds C represented by the formula (8) below and compounds D represented by the formula (9) below:

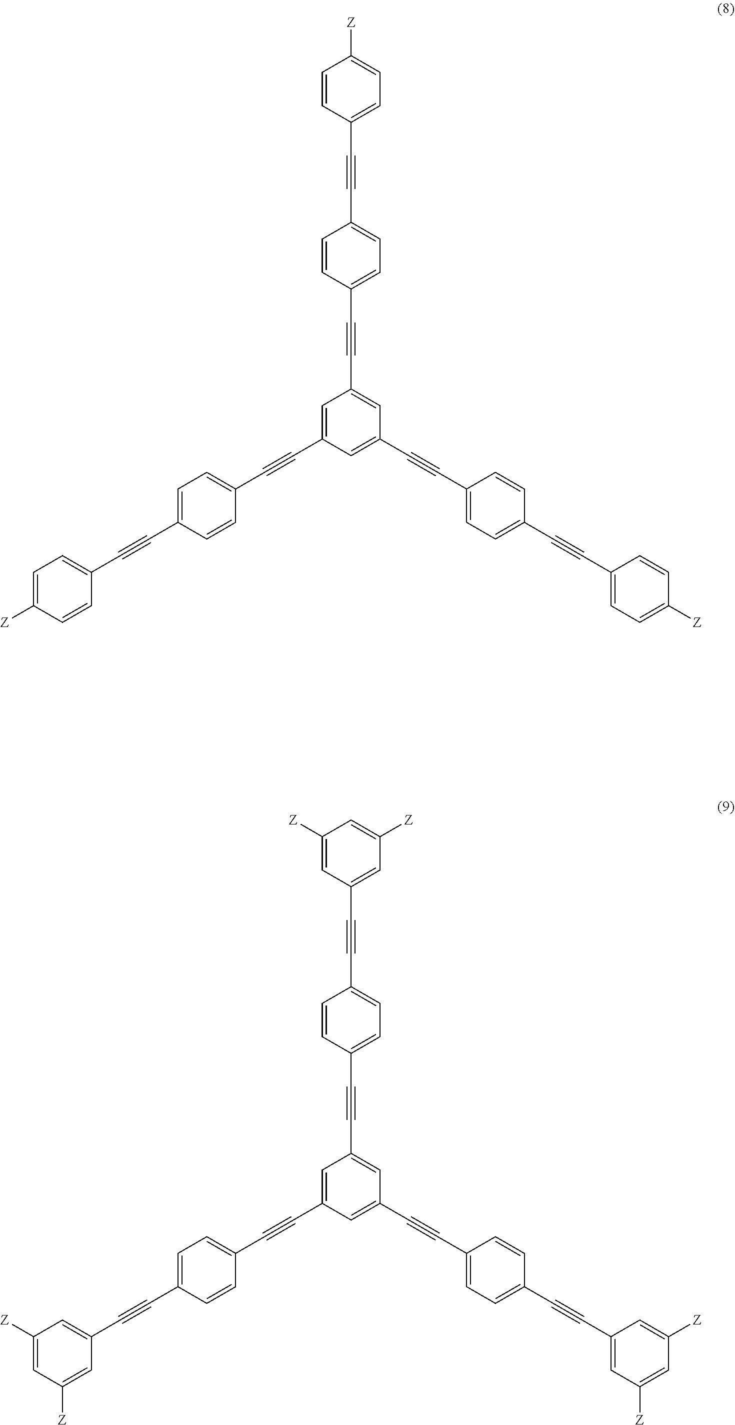

In the formula (8), the plurality of Z are the same as one another. The plurality of Z correspond to R¹, R⁶ and R¹¹, respectively, in the formula (5). The plurality of Z may be hydrogen atoms or substituents described in Table 1 below. In the formula (8), the plurality of Z may be —COOH, —COOC₄H₉ or —COOC₈H₁₇.

TABLE 1

| | Z |
|---|---|
| 1 | —H |
| 2 | —F |
| 3 | —CH₃ |
| 4 | —C₂H₅ |
| 5 | —CF₃ |
| 6 | —OH |
| 7 | —COOH |
| 8 | —COOCH₃ |
| 9 | —COOC₄H₉ |
| 10 | —COOC₈H₁₇ |
| 11 | —COCH₃ |
| 12 | —CONH₂ |
| 13 | —CN |
| 14 | —OCH₃ |
| 15 | —OCOCH₃ |
| 16 | —SH |
| 17 | —SO₃H |
| 18 | —SCOCH₃ |
| 19 | —SO₂CH₃ |
| 20 | —SO₂NH₂ |
| 21 | —NH₂ |
| 22 | —N(CH₃)₂ |
| 23 | —NO₂ |

In the formula (9), the plurality of Z are the same as one another. The plurality of Z correspond to R², R³, R⁷, R⁸, R¹² and R¹³, respectively, in the formula (5). The plurality of Z may be hydrogen atoms or substituents described in Table 1 above. In the formula (9), the plurality of Z may be —COOH, —COOC₄H₉ or —COOC₈H₁₇.

When the compound A is represented by the formula (5) below, at least one selected from the group consisting of R¹, R⁶ and R¹¹ may be a halogen atom, a C2 or higher alkyl group, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a primary amino group or a secondary amino group.

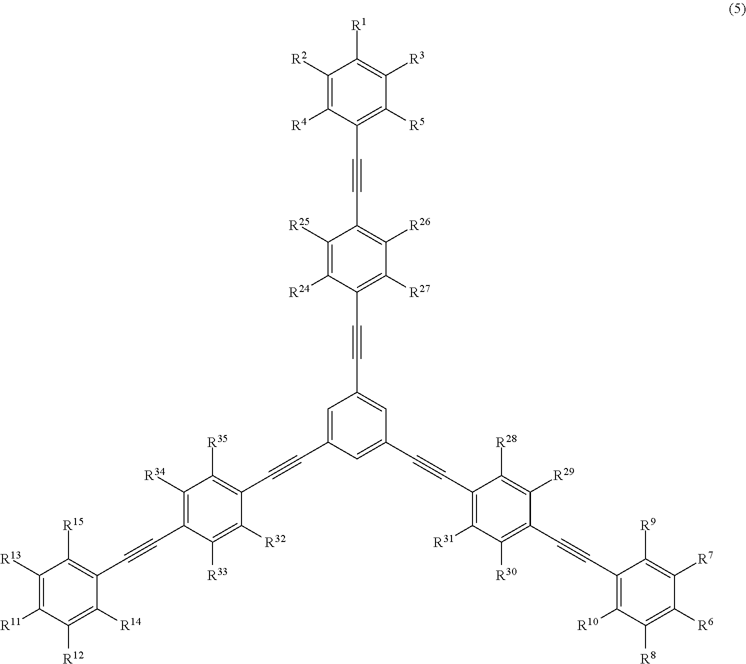

When the compound A is represented by the formula (17) below, at least one selected from the group consisting of R¹, R⁶ and R¹¹ may be a halogen atom, a C2 or higher alkyl group, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a silyl group, a primary amino group or a secondary amino group.

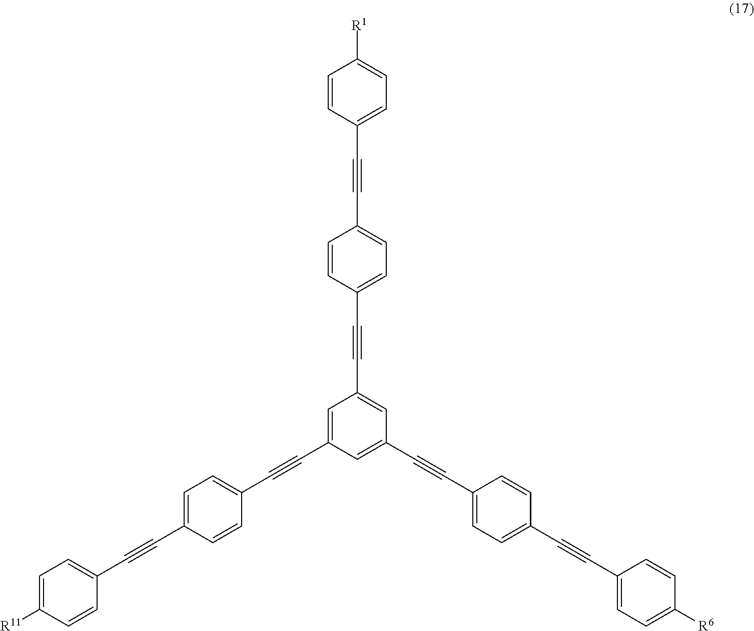

L¹ to L³ in the formula (1) may be each represented by the formula (3). The compound A may be, for example, compound E represented by the following formula (6):

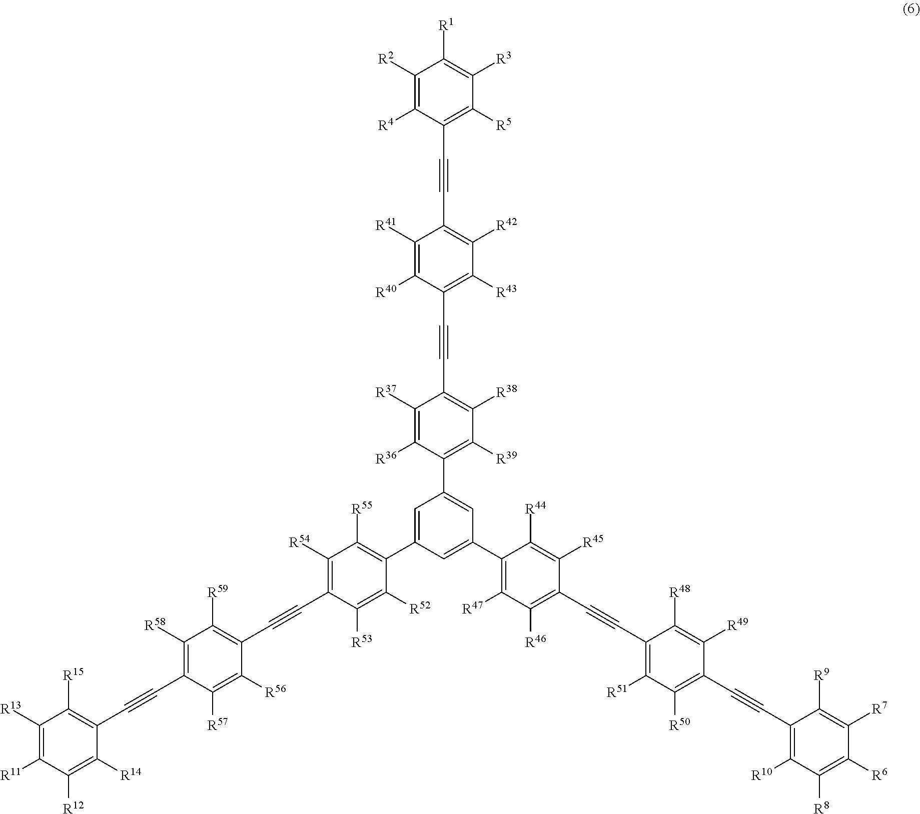

In the formula (6), R³⁶ to R⁵⁹ each independently include at least one atom selected from the group consisting of H, C, N, O, F, P, S, Cl, I and Br. Each of R³⁶ to R⁵⁹ corresponds to any of R²⁰ to R²³ described hereinabove.

Specific examples of the compounds E include compounds F represented by the formula (10) below and compounds G represented by the formula (11) below.

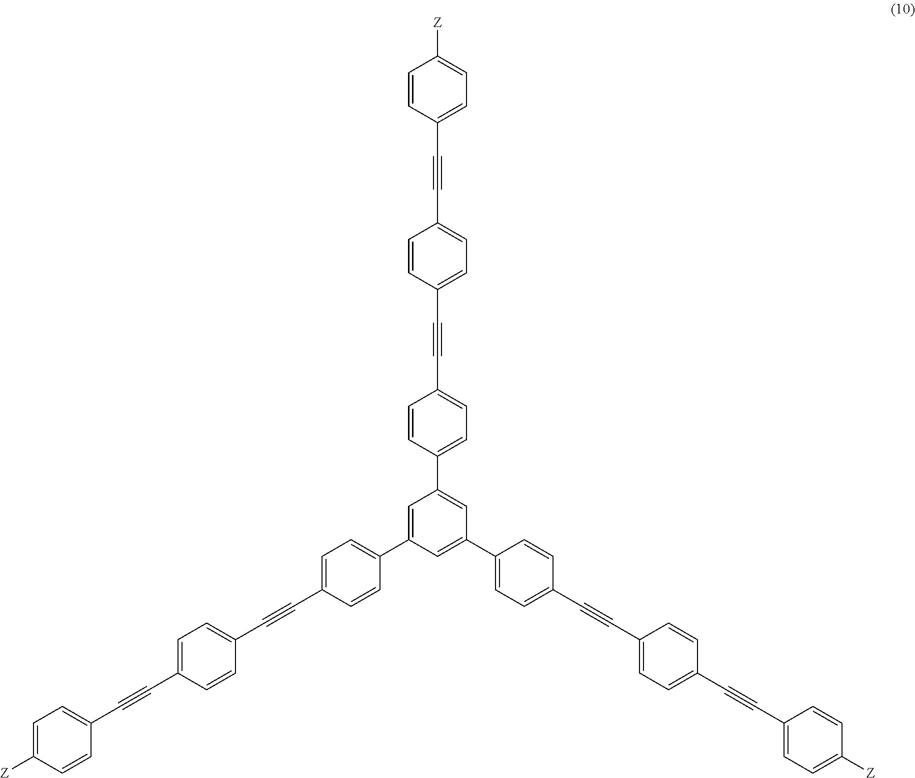

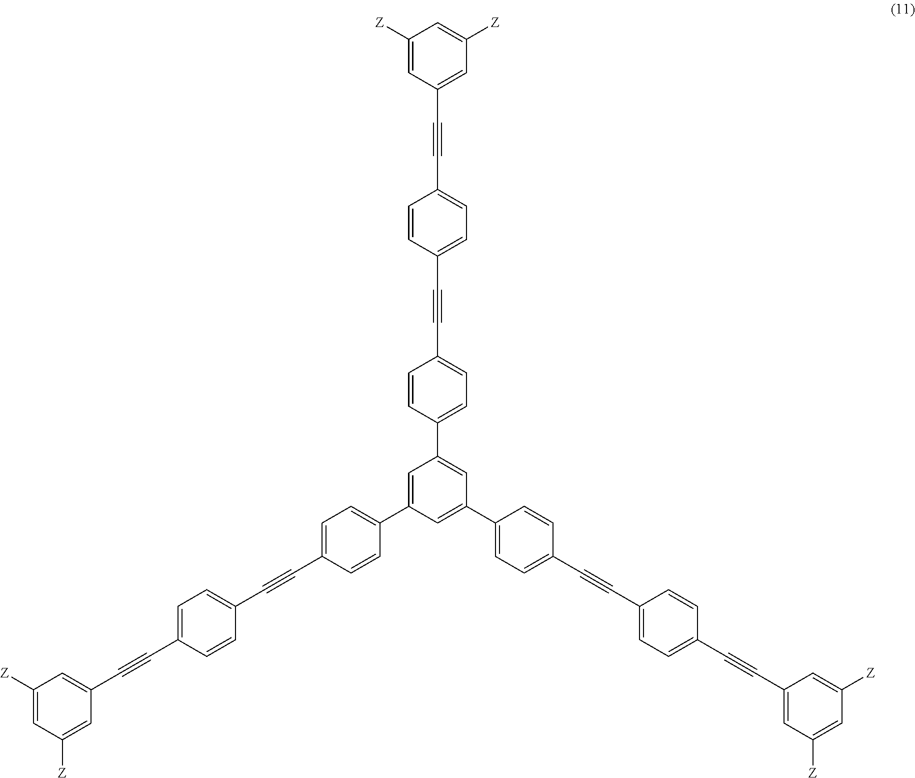

In the formula (10), the plurality of Z are the same as one another. The plurality of Z correspond to R¹, R⁶ and R¹¹, respectively, in the formula (6). The plurality of Z may be hydrogen atoms or substituents described in Table 1 hereinabove. In the formula (10), the plurality of Z may be —COOH, —COOC₄H₉ or —COOC₈H₁₇.

In the formula (11), the plurality of Z are the same as one another. The plurality of Z correspond to R², R³, R⁷, R⁸, R¹² and R¹³, respectively, in the formula (6). The plurality of Z may be hydrogen atoms or substituents described in Table 1 hereinabove.

The compound A may be, for example, compound H represented by the following formula (7):

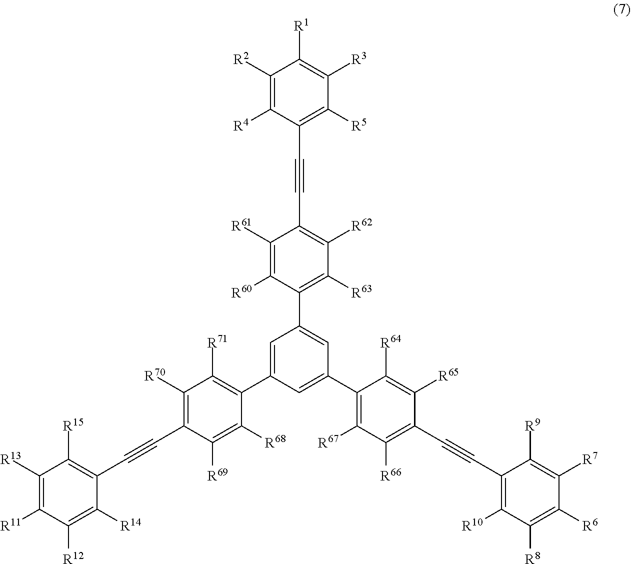

In the formula (7), R⁶⁰ to R⁷¹ each independently include at least one atom selected from the group consisting of H, C, N, O, F, P, S, Cl, I and Br. Each of R⁶⁰ to R⁷¹ corresponds to any of R²⁰ to R²³ described hereinabove.

When the compound A is compound I represented by the formula (4) below, at least one selected from the group consisting of R¹, R⁶ and R¹¹is a halogen atom, an alkyl group, an alkyl halide group, an unsaturated hydrocarbon group, a hydroxyl group, a carboxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group or a secondary amino group. In this case, at least one selected from the group consisting of R¹, R⁶ and R¹¹ may be a halogen atom, an alkyl group, an alkyl halide group, an unsaturated hydrocarbon group, a hydroxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group or a secondary amino group. However, in some cases, R¹, R⁶ and R¹¹ in the formula (4) may be each a hydrogen atom or a substituent other than those substituents described hereinabove.

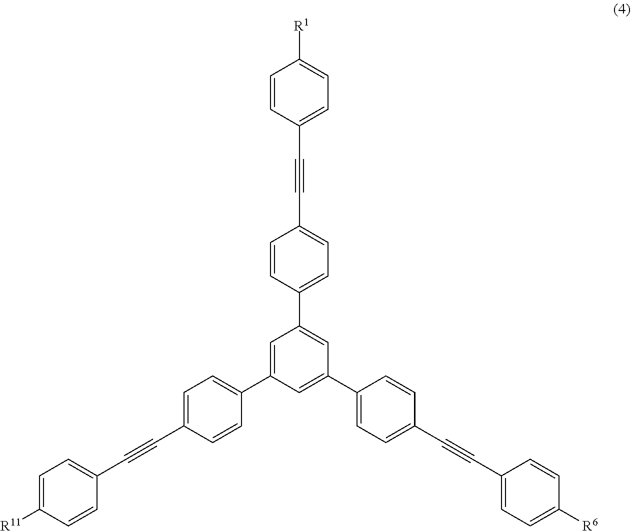

Specific examples of the compounds H represented by the formula (7) include compounds J represented by the formula (12) below and compounds K represented by the formula (13) below.

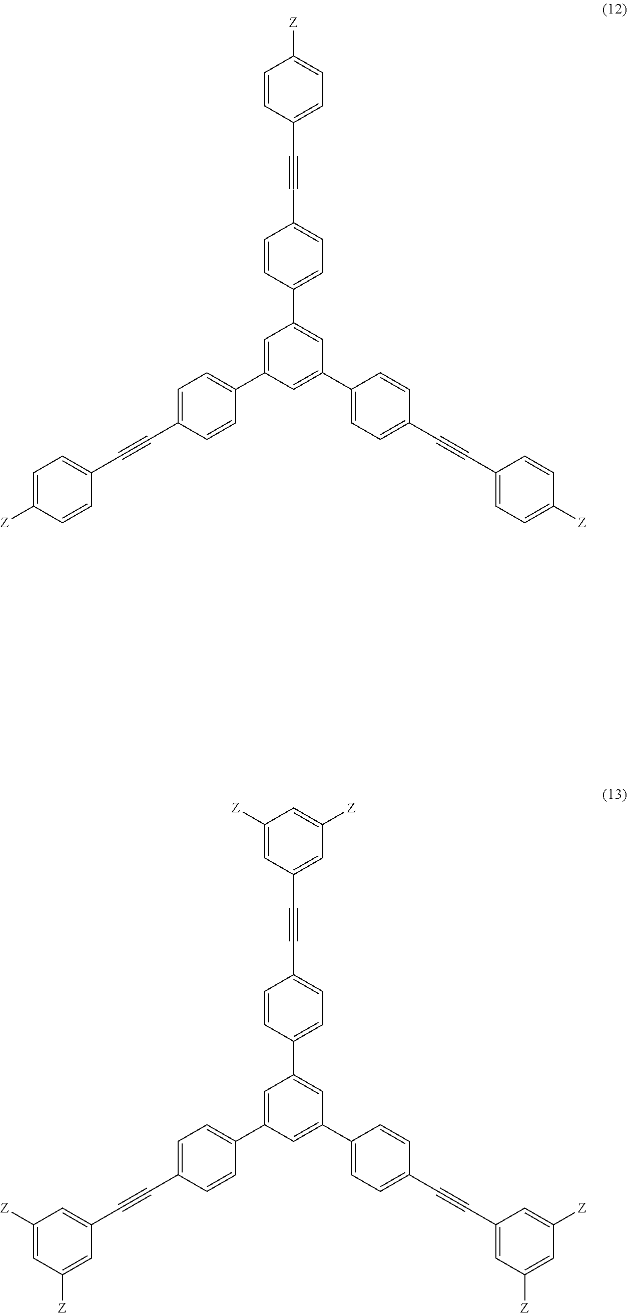

In the formula (12), the plurality of Z are the same as one another. The plurality of Z correspond to R¹, R⁶ and R¹¹, respectively, in the formula (4). The plurality of Z may be at least one selected from the substituents 2 to 12 and 15 to 21 described in Table 1 hereinabove. However, in some cases, the plurality of Z may be hydrogen atoms, the substituents 13, the substituents 14, the substituents 22 or the substituents 23 described in Table 1. In the formula (12), the plurality of Z may be —COOH, —COOC₄H₉ or —COOC₈H₁₇.

In the formula (13), the plurality of Z are the same as one another. The plurality of Z correspond to R², R³, R⁷, R⁸, R¹² and R¹³, respectively, in the formula (7). The plurality of Z may be hydrogen atoms or any of the substituents described in Table 1 hereinabove. In the formula (13), the plurality of Z may be —COOH, —COOC₄H₉ or —COOC₈H₁₇.

The compounds F represented by the formula (10), the compounds G represented by the formula (11), the compounds J represented by the formula (12) and the compounds K represented by the formula (13) may be synthesized by any methods without limitation. For example, the compounds F, G, J and K may be synthesized by the following method. First, compound L represented by the formula (14) below is provided.

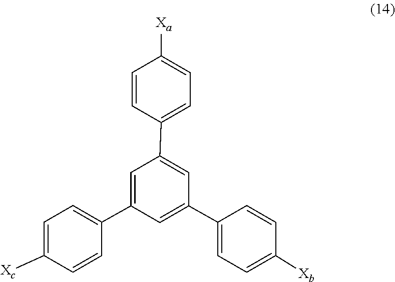

In the formula (14), Xₐ to X_{c} are each independently a substituent having reactivity in coupling reaction. Typical examples of such substituents are halogen groups. Xₐ to X_{c} may be ethynyl groups. Next, the compound L is subjected to coupling reaction with compound M having an appropriate structure to synthesize compound F, G, J or K.

The structure of the compound M is determined in accordance with the structure of the target compound. The coupling reaction conditions may be controlled appropriately in accordance with, for example, the structures of the compounds L and M.

The compounds C represented by the formula (8) and the compounds D represented by the formula (9) may be synthesized by any methods without limitation. For example, the compounds C and D may be synthesized by the following method. First, compound N represented by the formula (15) below is provided.

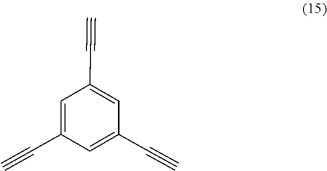

Next, the compound N is subjected to coupling reaction with compound O having an appropriate structure to synthesize compound C or D. The structure of the compound O is determined in accordance with the structure of the target compound. For example, the compound O contains a substituent having reactivity in coupling reaction. Typical examples of such substituents are halogen groups. The coupling reaction conditions may be controlled appropriately in accordance with, for example, the structures of the compounds N and O.

The compound A represented by the formula (1) has excellent two-photon absorption characteristics and low single-photon absorption characteristics when irradiated with light having a wavelength in the short wavelength region. In the present specification, the short wavelength region means a wavelength region including 405 nm, for example, a wavelength region of greater than or equal to 390 nm and less than or equal to 420 nm. When, for example, the compound A is irradiated with light having a wavelength of 405 nm, the compound A may exhibit two-photon absorption and substantially no single-photon absorption.

The two-photon absorption cross section of the compound A irradiated with light having a wavelength of 405 nm may be greater than 500 GM, or may be greater than or equal to 1000 GM, greater than or equal to 1500 GM, or greater than or equal to 2000 GM. The upper limit of the two-photon absorption cross section of the compound A is not particularly limited, and is, for example, 5000 GM. For example, the two-photon absorption cross section may be measured by the z-scan technique described in J. Opt. Soc. Am. B, 2003, Vol. 20, p. 529. The z-scan technique is widely used as a method for measuring non-linear optical constants. In the z-scan technique, a measurement sample is moved along the beam irradiation direction near the focal point at which the laser beam is focused. During this process, the change in the amount of light transmitted through the measurement sample is recorded. In the z-scan technique, the power density of incident light changes depending on the location of the measurement sample. Thus, when the measurement sample absorbs light non-linearly, the amount of transmitted light is attenuated when the measurement sample is located near the focal point of the laser beam. The two-photon absorption cross section may be calculated by fitting the changes in the amount of transmitted light based on the theoretical curve predicted from conditions such as the intensity of the incident light, the thickness of the measurement sample, and the concentration of the compound A in the measurement sample.

The two-photon absorption cross section may be a value calculated by computational chemistry. Several methods have been proposed for estimating the two-photon absorption cross section by computational chemistry. For example, the two-photon absorption cross section may be calculated based on the quadratic non-linear response theory described in J. Chem. Theory Comput. 2018, Vol. 14, p. 807.

The molar absorption coefficient of the compound A with respect to light having a wavelength of 405 nm may be less than or equal to 650 L/(mol·cm), less than or equal to 500 L/(mol·cm), less than or equal to 250 L/(mol·cm), or less than or equal to 100 L/(mol·cm). The lower limit of the molar absorption coefficient of the compound A is not particularly limited, and is, for example, 0.01 L/(mol·cm). The molar absorption coefficient may be measured by, for example, a method in accordance with the manual specified in Japanese Industrial Standards (JIS) K0115: 2004. In the measurement of the molar absorption coefficient, a light source is used that emits light with such a photon density that the compound A does not substantially exhibit two-photon absorption. The molar absorption coefficient may be used as an index of single-photon absorption.

The molar absorption coefficient may be a value calculated with a quantum chemistry calculation program. For example, Gaussian 16 (manufactured by Gaussian) may be used as the quantum chemistry calculation program.

In two-photon absorption, the compound A absorbs about twice as much energy as the energy of light applied to the compound A. The wavelength of light having about twice the energy of 405 nm wavelength light is, for example, 200 nm. That is, when the compound A is irradiated with light having a wavelength of about 200 nm, single-photon absorption may occur in the compound A. Further, single-photon absorption may occur in the compound A when the compound is irradiated with light having a wavelength close to the wavelength region causing two-photon absorption.

The compound A also tends to have a high fluorescence quantum yield. In the present specification, specifically, the term “quantum yield” means internal quantum yield. The wavelength of the fluorescent light emitted by the compound A may be greater than or equal to 405 nm and less than or equal to 660 nm, and in some cases may be greater than or equal to 350 nm and less than or equal to 650 nm. The fluorescence quantum yield of the compound A may be greater than or equal to 35%, greater than or equal to 40%, or greater than or equal to 50%. The upper limit of the fluorescence quantum yield of the compound A is not particularly limited, and is, for example, 99%. The fluorescence quantum yield may be measured with, for example, a commercially available absolute PL quantum yield measuring device.

The light-absorbing material of the present embodiment may include compound A represented by the formula (1) as a main component. The term “main component” means that the component represents the highest weight ratio among the components contained in the light-absorbing material. For example, the light-absorbing material consists essentially of compound A. The phrase “consist essentially of” means that other components that will alter the essential characteristics of the material mentioned are excluded. However, the light-absorbing material may contain impurities in addition to the compound A.

The light-absorbing material of the present embodiment functions as, for example, a multiphoton absorption material such as a two-photon absorption material. In particular, by virtue of including the compound A represented by the formula (1), the light-absorbing material of the present embodiment exhibits highly non-linear two-photon absorption characteristics when irradiated with light having a wavelength in the short wavelength region.

For example, the light-absorbing material of the present embodiment is used in a device that utilizes light having a wavelength in the short wavelength region. Examples of such devices include recording media, modeling machines and fluorescence microscopes. Examples of the recording media include three-dimensional optical memories. Specific examples of the three-dimensional optical memories include three-dimensional optical discs. Examples of the modeling machines include optical modeling machines such as 3D printers. Examples of the fluorescence microscopes include two-photon fluorescence microscopes. The light utilized in these devices has a high photon density at, for example, near the focal point. The power density near the focal point of the light used in the device is, for example, greater than or equal to 0.1 W/cm² and less than or equal to 1.0×10²⁰ W/cm². The power density near the focal point of the light may be greater than or equal to 1.0 W/cm², greater than or equal to 1.0×10² W/cm², or greater than or equal to 1.0×10⁵ W/cm². For example, a femtosecond laser such as a Ti:sapphire laser may be used as the light source of the device.

That is, in another aspect, the present disclosure provides a light-absorbing material used in a device utilizing light having a wavelength of greater than or equal to 390 nm and less than or equal to 420 nm, the light-absorbing material including a compound represented by the following formula (1):

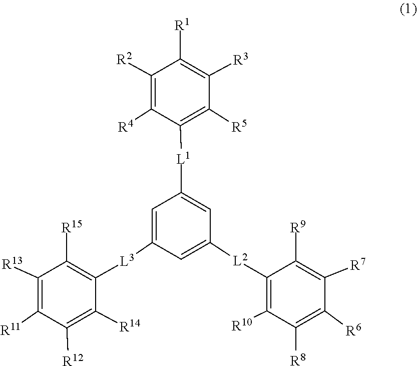

wherein R¹ to R¹⁵ each independently include at least one atom selected from the group consisting of H, C, N, O, F, P, S, Cl, I and Br, and L¹ to L³ are each independently represented by the following formula (2) or (3):

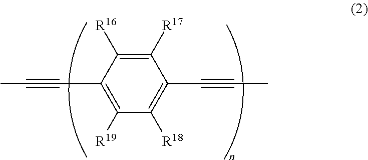

wherein R¹⁶ to R¹⁹ each independently include at least one atom selected from the group consisting of H, C, N, O, F, P, S, Cl, I and Br, and n is an integer of 1 to 3,

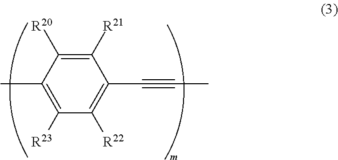

wherein R²⁰ to R²³ each independently include at least one atom selected from the group consisting of H, C, N, O, F, P, S, Cl, I and Br, and m is an integer of 1 to 3.

Recording media include a thin film called, for example, a recording layer or a recording film. In a recording medium, information is recorded on the recording layer or the recording film. As an example, a thin film as a recording layer or a recording film includes the light-absorbing material of the present embodiment.

That is, in another aspect, the present disclosure provides a recording medium that includes a recording film including a light-absorbing material including a compound represented by the following formula (1):

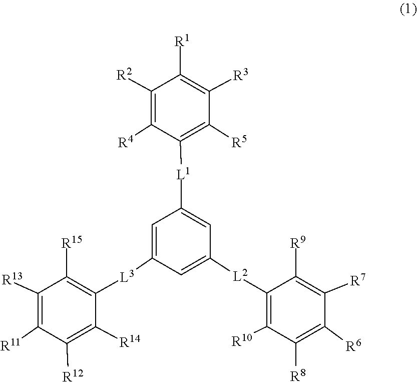

wherein R¹ to R¹⁵ each independently include at least one atom selected from the group consisting of H, C, N, O, F, P, S, Cl, I and Br, and L¹ to L³ are each independently represented by the following formula (2) or (3):

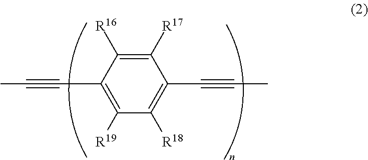

wherein R¹⁶ to R¹⁹ each independently include at least one atom selected from the group consisting of H, C, N, O, F, P, S, Cl, I and Br, and n is an integer of 1 to 3,

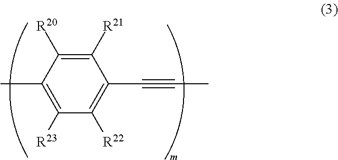

wherein R²⁰ to R²³ each independently include at least one atom selected from the group consisting of H, C, N, O, F, P, S, Cl, I and Br, and m is an integer of 1 to 3.

The recording medium may include a dielectric layer in addition to the recording layer. For example, the recording medium includes a plurality of recording layers and a plurality of dielectric layers. In the recording medium, a plurality of recording layers and a plurality of dielectric layers may be alternately stacked on top of one another.

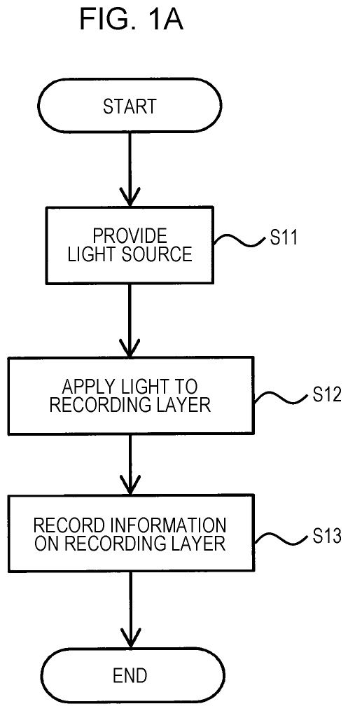

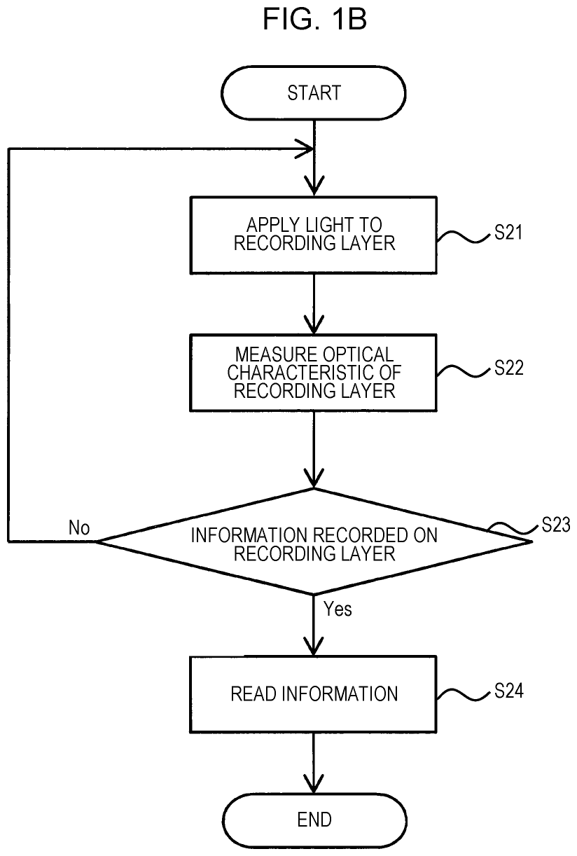

Next, a method for recording information using the recording medium described above will be described. FIG. 1A is a flowchart regarding an information recording method using the recording medium described above. First, in step S11, a light source is provided that emits light having a wavelength of greater than or equal to 390 nm and less than or equal to 420 nm. For example, a femtosecond laser such as a Ti:sapphire laser may be used as the light source. Next, in step S12, the light from the light source is focused with a lens and is applied to a recording layer in the recording medium. Specifically, the light from the light source is focused with a lens or other device and is applied to a recording region in the recording medium. The power density near the focal point of the light is, for example, greater than or equal to 0.1 W/cm² and less than or equal to 1.0×10²⁰ W/cm². The power density near the focal point of the light may be greater than or equal to 1.0 W/cm², greater than or equal to 1.0×10² W/cm², or greater than or equal to 1.0×10⁵ W/cm². In the present specification, the recording region means a spot that is present in the recording layer and, by being irradiated with light, allows information to be recorded therein.

In the recording region that has been irradiated with the light, a physical change or a chemical change occurs to give rise to a change in optical characteristics of the recording region. For example, the intensity of fluorescent light emitted from the recording region is lowered. In this manner, information can be recorded in the recording layer, specifically, the recording region (step S13).

That is, in another aspect, the present disclosure provides:
an information recording method including:
   providing a light source that emits light having a wavelength of greater than or equal to 390 nm and less than or equal to 420 nm; and
   focusing the light from the light source with a lens and applying the light to a recording region in a recording medium including a light-absorbing material including a compound represented by the following formula (1):

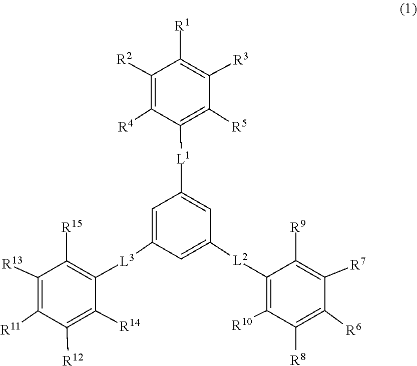

wherein R¹ to R¹⁵ each independently include at least one atom selected from the group consisting of H, C, N, O, F, P, S, Cl, I and Br, and L¹ to L³ are each independently represented by the following formula (2) or (3):

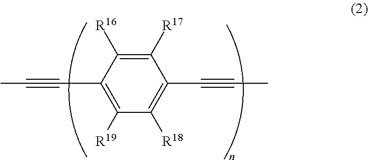

wherein R¹⁶ to R¹⁹ each independently include at least one atom selected from the group consisting of H, C, N, O, F, P, S, Cl, I and Br, and n is an integer of 1 to 3,

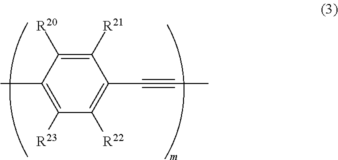

wherein R²⁰ to R²³ each independently include at least one atom selected from the group consisting of H, C, N, O, F, P, S, Cl, I and Br, and m is an integer of 1 to 3.

Next, a method for reading information using the recording medium described hereinabove will be described. FIG. 1B is a flowchart regarding an information reading method using the recording medium described hereinabove. First, in step S21, light is applied to a recording layer in the recording medium. Specifically, light is applied to a recording region in the recording medium. The light used in step S21 may be the same as or different from the light used to record information on the recording medium. Next, in step S22, optical characteristics of the recording layer are measured. Specifically, optical characteristics of the recording region are measured. In step S22, for example, the intensity of fluorescent light emitted from the recording region is measured.

Next, in step S23, judgement is made as to whether information has been recorded in the recording layer, based on the optical characteristics of the recording layer. For example, the recording layer is judged to store information when the intensity of fluorescent light emitted from the recording region is less than or equal to a predetermined value. When, on the other hand, the intensity of fluorescent light is more than a predetermined value, the recording layer is judged to store no information. When the judgement is that no information is recorded in the recording region, the process returns to step S21 and performs the same operation on other recording layer. When any recording layer is judged to store information, the information is read out in step S24.

For example, the methods described above for recording and reading information using the recording medium may be performed with a known recording device. For example, the recording device includes a light source that applies light to a recording region in the recording medium, a measuring device that measures optical characteristics of the recording region, and a controller that controls the light source and the measuring device.

Modeling machines form shapes by, for example, irradiating a photocurable resin composition with light to cure the resin composition. As an example, a photocurable resin composition for stereolithography includes the light-absorbing material of the present embodiment. The photocurable resin composition usually includes a polymerizable compound and a polymerization initiator in addition to the light-absorbing material. The photocurable resin composition may further include an additive such as a binder resin. The photocurable resin composition may include an epoxy resin.

A fluorescence microscope applies light to, for example, a biological sample containing a fluorescent dye material and allows for observation of fluorescence emitted from the dye material. As an example, a fluorescent dye material that is added to a biological sample includes the light-absorbing material of the present embodiment. Examples

Hereinbelow, the present disclosure will be described in greater detail by way of Examples. Examples discussed below are only illustrative and do not limit the scope of the present disclosure. In the present disclosure, the compounds used in Examples are written as “compound (X)—Y”. “X” indicates the structural formula of the compound. “Y” indicates the type of Z in the formula (X). For example, compound (12)-7 means a compound represented by the formula (12) in which Z is substituent 7 (—COOH) described in Table 1.

### Synthesis of Compound (12)-1

First, 4′-bromoacetophenone and ethynylbenzene were dissolved into a mixed solution of tetrahydrofuran and triethylamine. Catalytic amounts of [1,1′-bis(diphenylphosphino)ferrocene] dichloropalladium (II) and copper (I) iodide were further added to the solution. Next, the solution was stirred at room temperature for 16 hours. Hydrochloric acid was added to the resultant reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give a precursor of compound (12)-1.

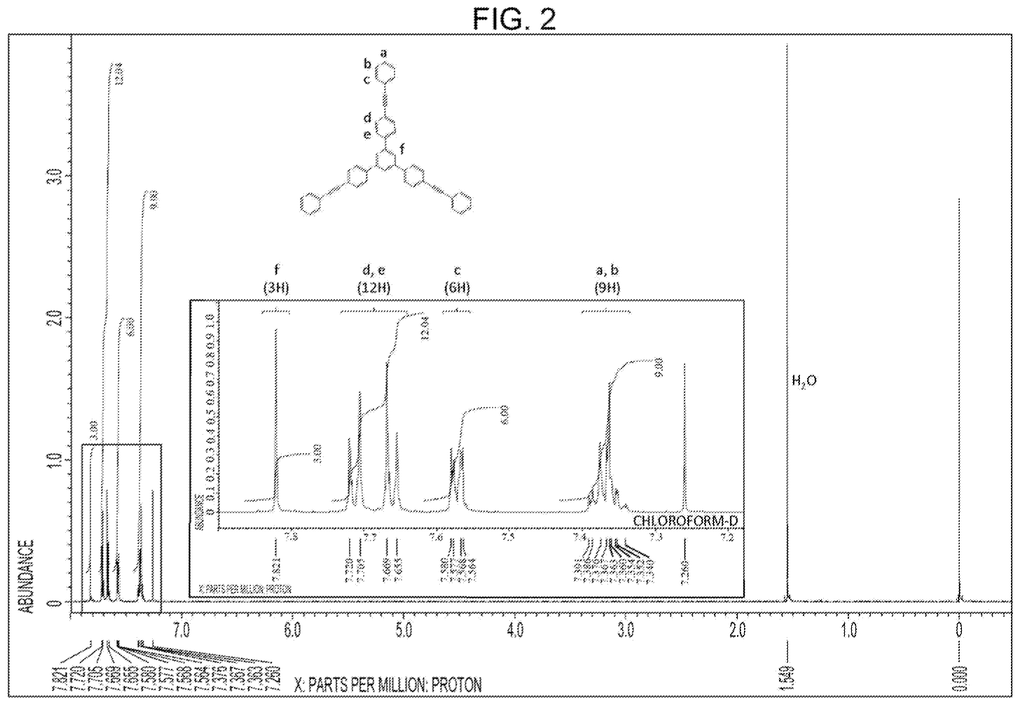

Next, the precursor of compound (12)-1 and phosphotungstic acid hydrate were mixed. The mixture was heated under reflux overnight while performing stirring. After the reaction in the solution had completed, the solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (12)-1 as a white solid. Compound (12)-1 was identified by ¹H-NMR. FIG. 2 is a graph illustrating the ¹H-NMR spectrum of compound (12)-1. The ¹H-NMR spectrum of compound (12)-1 was as follows.

¹H-NMR (600 MHz, CHLOROFORM-D) δ 6 7.82 (s, 3H), 7.69 (dd, J=30.6, 8.6 Hz, 12H), 7.57 (dd, J=7.6, 2.1 Hz, 6H), 7.34-7.39 (m, 9H).

### Synthesis of Compound (12)-7

First, 1,3,5-tris[4′-(ethynyl)phenyl]benzene and methyl 4-iodobenzoate were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the resultant reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give a precursor of compound (12)-7.

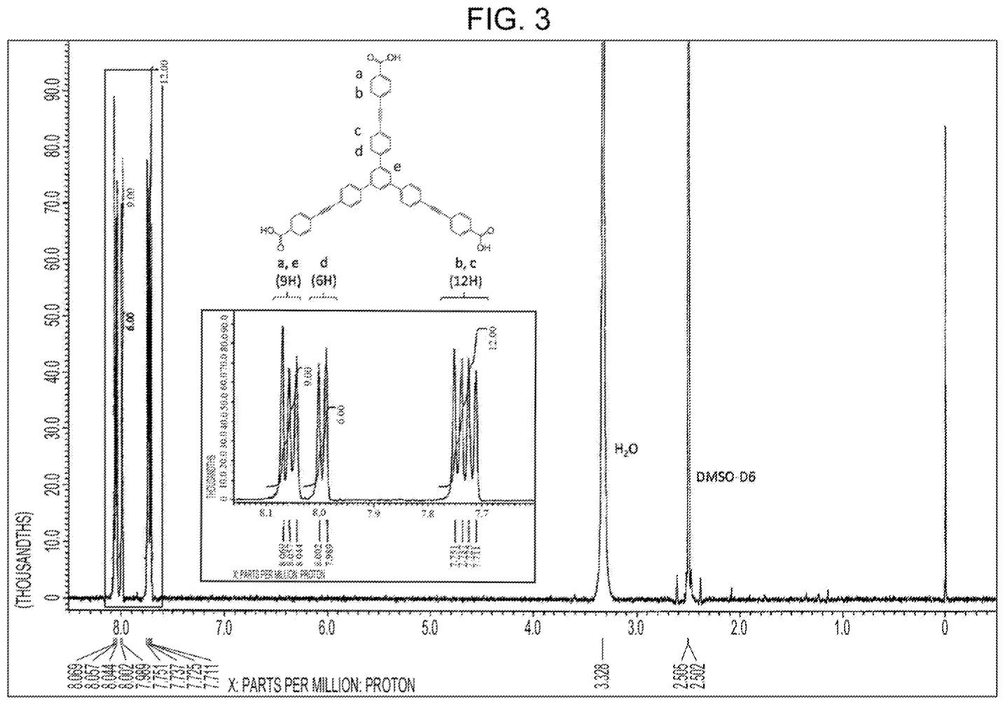

Next, the precursor of compound (12)-7 was dissolved into a mixed solution of tetrahydrofuran and methanol (v/v=1:1). An aqueous sodium hydroxide solution was added to the solution. The mixture was heated under reflux overnight while performing stirring. After the reaction in the solution had completed, dilute hydrochloric acid was added to the solution. This addition acidified the solution and caused a solid to precipitate. The solid was washed with pure water to give compound (12)-7 as a white solid. Compound (12)-7 was identified by ¹H-NMR. FIG. 3 is a graph illustrating the ¹H-NMR spectrum of compound (12)-7. The ¹H-NMR spectrum of compound (12)-7 was as follows.

¹H-NMR (600 MHz, DMSO-D6) δ 8.06 (t, J=7.6 Hz, 9H), 8.00 (d, J=8.3 Hz, 6H), 7.73 (q, J=8.0 Hz, 12H).

### Synthesis of Compound (12)-9

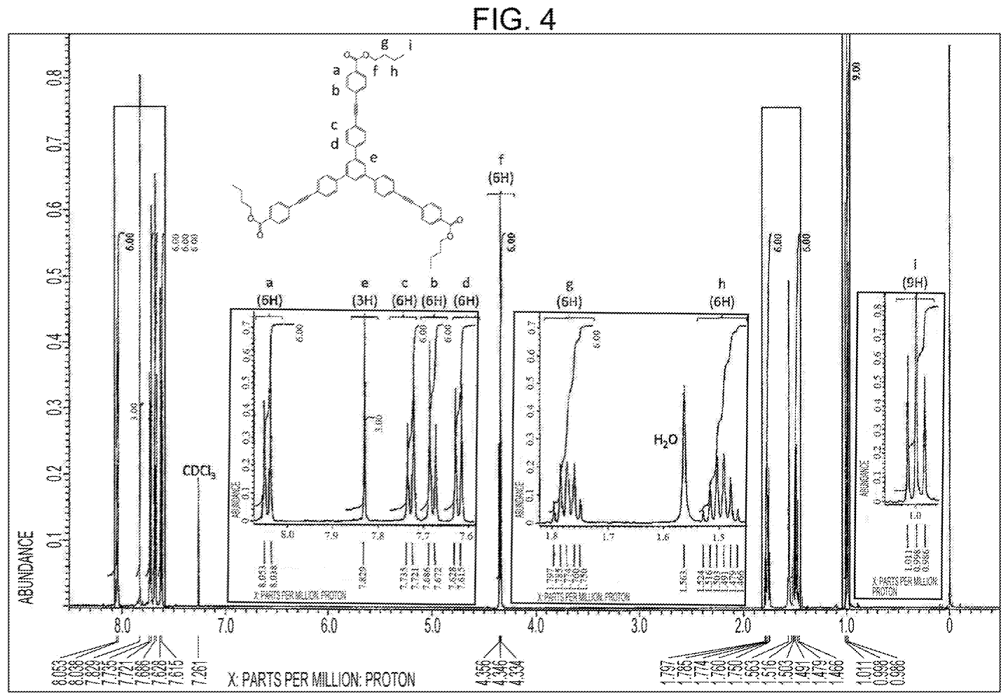

First, compound (12)-7 described above was added to a butanol solvent to form a suspension. Next, thionyl chloride was added to the suspension, and the mixture was heated under reflux overnight while performing stirring. A white solid was filtered off from the resultant reaction solution and was washed with methanol. The solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (12)-9 as a white solid. Compound (12)-9 was identified by ¹H-NMR. FIG. 4 is a graph illustrating the ¹H-NMR spectrum of compound (12)-9. The ¹H-NMR spectrum of compound (12)-9 was as follows.

¹H-NMR (600 MHz, CHLOROFORM-D) δ 6 8.05 (d, J=9.0 Hz, 6H), 7.83 (s, 3H), 7.73 (d, J=8.3 Hz, 6H), 7.68 (d, J=8.3 Hz, 6H), 7.62 (d, J=8.3 Hz, 6H), 4.35 (t, J=6.5 Hz, 6H), 1.75-1.80 (m, 6H), 1.50 (td, J=14.8, 7.3 Hz, 6H), 1.00 (t, J=7.6 Hz, 9H).

### Synthesis of Compound (12)-10

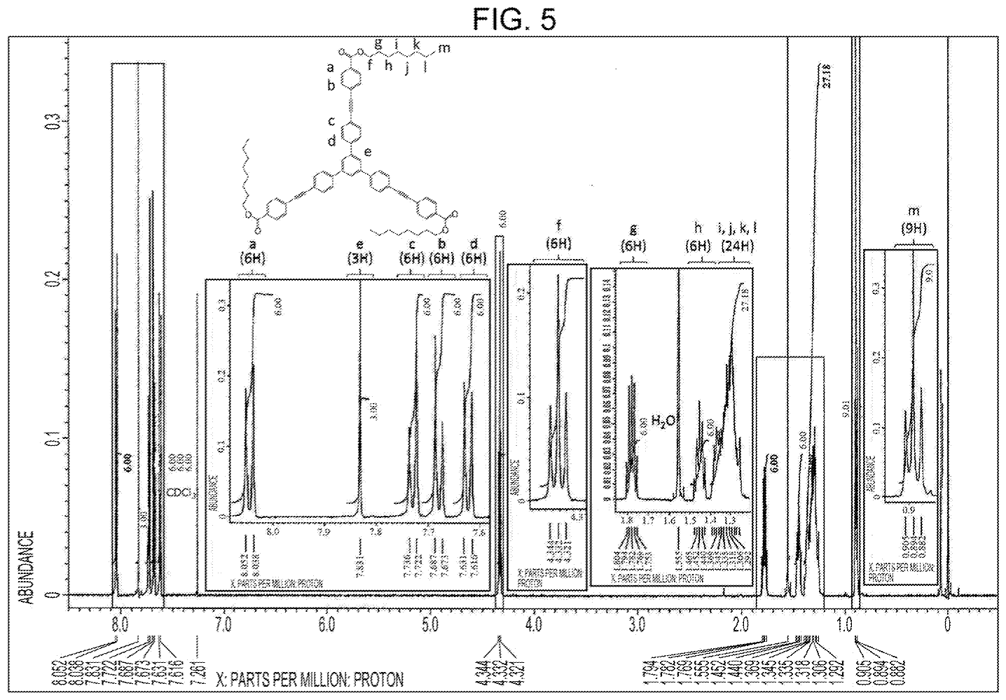

First, compound (12)-7 described above was added to an octanol solvent to form a suspension. Next, thionyl chloride was added to the suspension, and the mixture was heated under reflux overnight while performing stirring. A white solid was filtered off from the resultant reaction solution and was washed with methanol. The solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (12)-10 as a white solid. Compound (12)-10 was identified by ¹H-NMR. FIG. 5 is a graph illustrating the ¹H-NMR spectrum of compound (12)-10. The ¹H-NMR spectrum of compound (12)-10 was as follows.

¹H-NMR (600 MHz, CHLOROFORM-D) δ 8.04 (d, J=8.3 Hz, 6H), 7.83 (s, 3H), 7.73 (d, J=8.3 Hz, 6H), 7.68 (d, J=8.3 Hz, 6H), 7.62 (d, J=9.0 Hz, 6H), 4.33 (t, J=6.9 Hz, 6H), 1.76-1.81 (m, 6H), 1.43-1.48 (m, 6H), 1.26-1.39 (m, 24H), 0.89 (t, J=6.9 Hz, 9H).

### Synthesis of Compound (13)-7

First, 1,3,5-tris(4-iodophenyl)benzene and dimethyl 5-iodoisophthalate were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give a precursor of compound (13)-7.

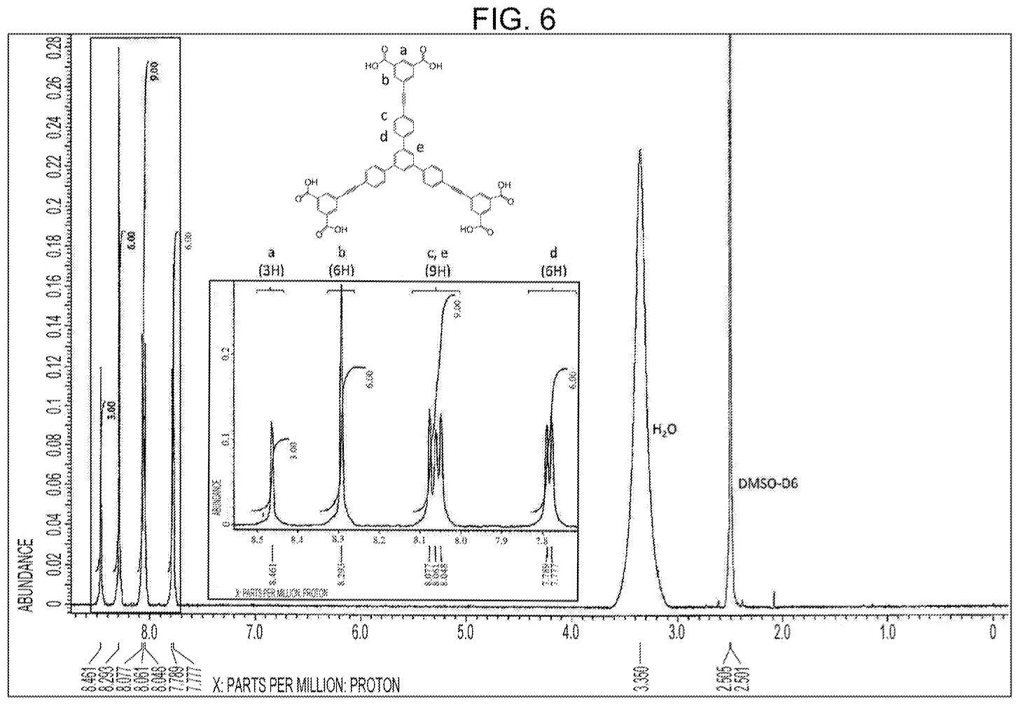

Next, the precursor of compound (13)-7 was dissolved into a mixed solution of tetrahydrofuran and methanol (v/v=1:1). An aqueous sodium hydroxide solution was added to the solution. The mixture was heated under reflux overnight while performing stirring. After the reaction in the solution had completed, dilute hydrochloric acid was added to the solution. This addition acidified the solution and caused a solid to precipitate. The solid was washed with pure water to give compound (13)-7 as a white solid. Compound (13)-7 was identified by ¹H-NMR. FIG. 6 is a graph illustrating the ¹H-NMR spectrum of compound (13)-7. The ¹H-NMR spectrum of compound (13)-7 was as follows.

¹H-NMR (600 MHz, DMSO-D6) δ 8.46 (s, 3H), 8.29 (s, 6H), 8.06 (t, J=8.6 Hz, 9H), 7.78 (d, J=6.9 Hz, 6H).

### Synthesis of Compound (13)-10

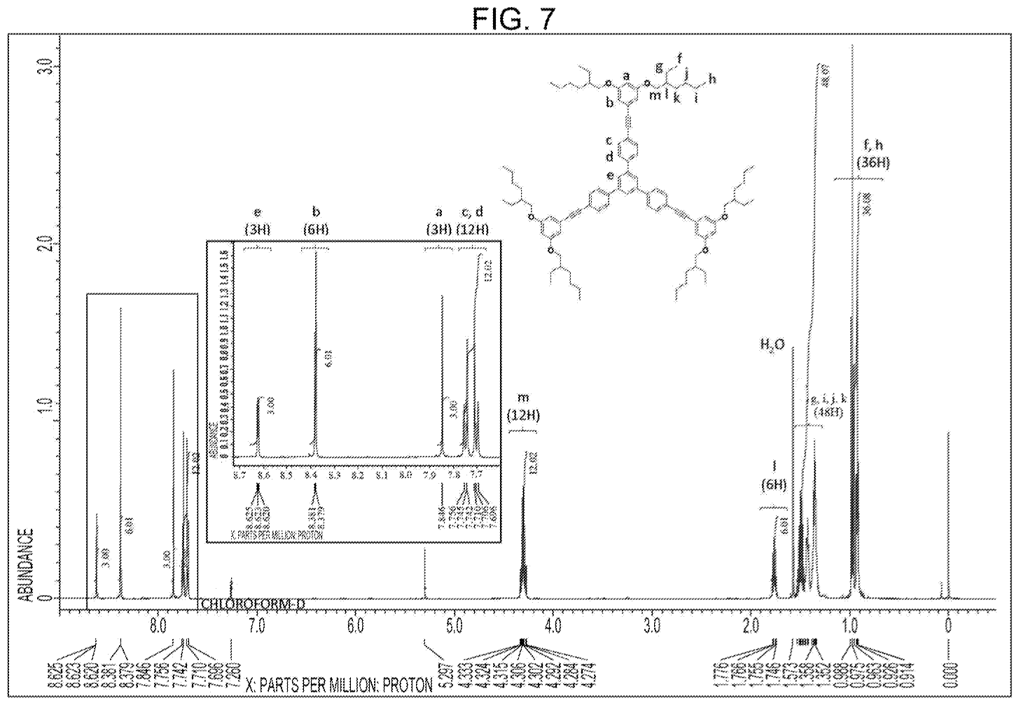

Compound (13)-7 described above was added to a 2-ethyl-1l-hexanol solvent to form a suspension. Next, sulfuric acid was added to the suspension, and the mixture was heated under reflux for 12 hours while performing stirring. The solvent was removed from the reaction solution, and the residue was extracted with ethyl acetate. The extract was purified by silica gel column chromatography to give compound (13)-10 as a white solid. Compound (13)-10 was identified by ¹H-NMR. FIG. 7 is a graph illustrating the ¹H-NMR spectrum of compound (13)-10. The ¹H-NMR spectrum of compound (13)-10 was as follows.

¹H-NMR (600 MHz, CHLOROFORM-D) δ 8.62 (t, J=1.7 Hz, 3H), 8.38 (d, J=1.4 Hz, 6H), 7.85 (s, 3H), 7.70-7.76 (m, 12H), 4.30 (dq, J=18.9, 5.6 Hz, 12H), 1.76 (q, J=6.0 Hz, 6H), 1.33-1.53 (m, 48H), 0.91-0.99 (m, 36H).

### Synthesis of Compound (8)-5

First, (4-iodophenylethynyl)trimethylsilane and 1-ethynyl-4-(trifluoromethyl)benzene were dissolved into tetrahydrofuran. To the solution were added N,N-diisopropylethylamine and catalytic amounts of triphenylphosphine, bis(triphenylphosphine)palladium (II) dichloride and copper (I) iodide. Next, the solution was stirred at room temperature for 3 days. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give precursor A of compound (8)-5.

Next, the precursor A of compound (8)-5 and tetrabutylammonium fluoride were dissolved into tetrahydrofuran. Next, the solution was stirred for 3 hours. Saturated aqueous sodium hydrogen carbonate solution was added to the reaction solution. Next, the reaction solution was extracted with ethyl acetate. The extract was washed with saturated brine. Precursor B of compound (8)-5 was thus obtained.

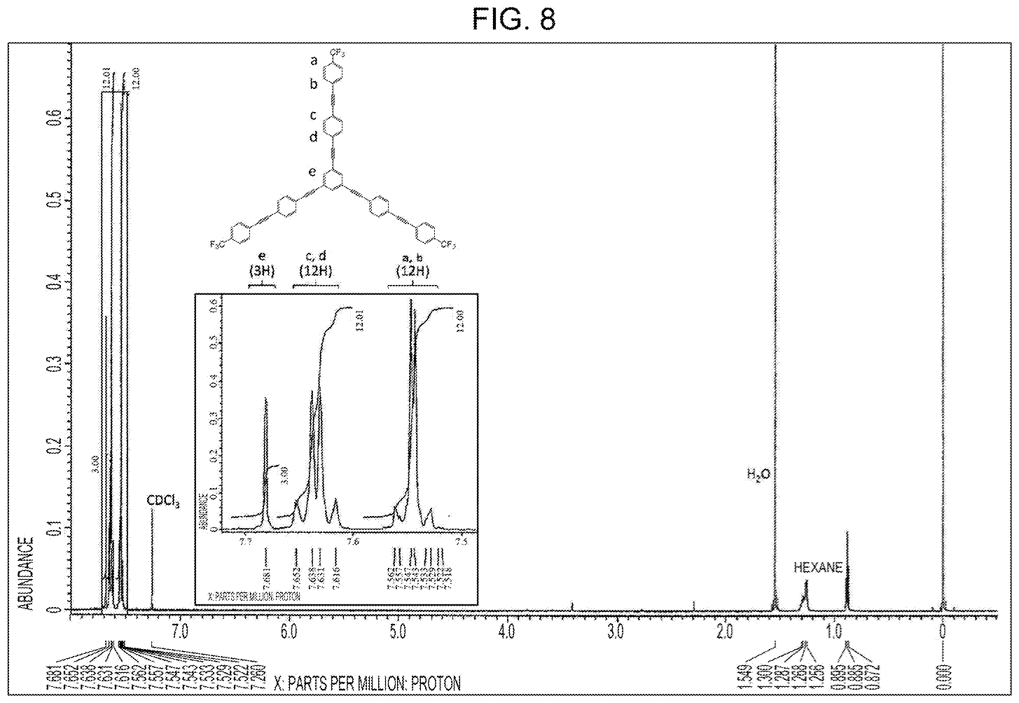

Next, the precursor B of compound (8)-5 and 1,3,5-tribromobenzene were dissolved into a mixed solution of diisopropylamine and 1,4-dioxane. Catalytic amounts of 2-dicyclohexylphosphino-2′,4′,6′-triisopropylbiphenyl, bis(acetonitrile)palladium (II) dichloride and copper (I) iodide were further added to the solution. Next, the solution was stirred at 80° C. for 20 hours. Hydrochloric acid was added to the reaction solution for neutralization. Next, the reaction solution was extracted with ethyl acetate. Sodium sulfate was added to the extract, and the extract was dehydrated. Next, the sodium sulfate was filtered off from the extract. The filtrate thus obtained was concentrated using a rotary evaporator. The concentrate thus obtained was purified by silica gel column chromatography to give compound (8)-5. FIG. 8 is a graph illustrating the ¹H-NMR spectrum of compound (8)-5. The ¹H-NMR spectrum of compound (8)-5 was as follows. ¹H-NMR (600 MHz, CHLOROFORM-D) δ 7.68 (s, 3H), 7.63 (dd, J=13.1, 9.0 Hz, 12H), 7.56-7.52 (m, 12H).

### Synthesis of Compound (8)-7

First, 1,3,5-triethynylbenzene and 1-ethynyl-4-iodobenzene were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give precursor A of compound (8)-7.

Next, the precursor A of compound (8)-7 and methyl 4-iodobenzoate were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give precursor B of compound (8)-7.

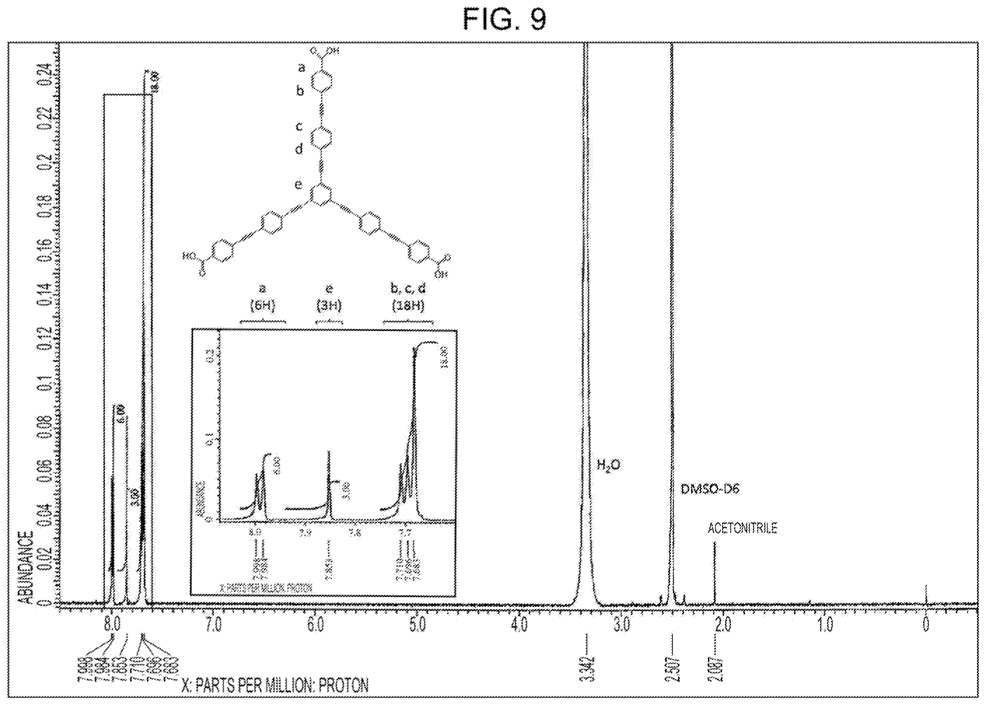

Next, the precursor B of compound (8)-7 was dissolved into a mixed solution of tetrahydrofuran and methanol (v/v=1:1). An aqueous sodium hydroxide solution was added to the solution. The mixture was heated under reflux overnight while performing stirring. After the reaction in the solution had completed, dilute hydrochloric acid was added to the solution. This addition acidified the solution and caused a solid to precipitate. The solid was washed with pure water to give compound (8)-7 as a white solid. Compound (8)-7 was identified by ¹H-NMR. FIG. 9 is a graph illustrating the ¹H-NMR spectrum of compound (8)-7. The ¹H-NMR spectrum of compound (8)-7 was as follows. ¹H-NMR (600 MHz, DMSO-D6) δ 7.99 (d, J=8.3 Hz, 6H), 7.85 (s, 3H), 7.70 (t, J=7.9 Hz, 18H).

### Synthesis of Compound (8)-9

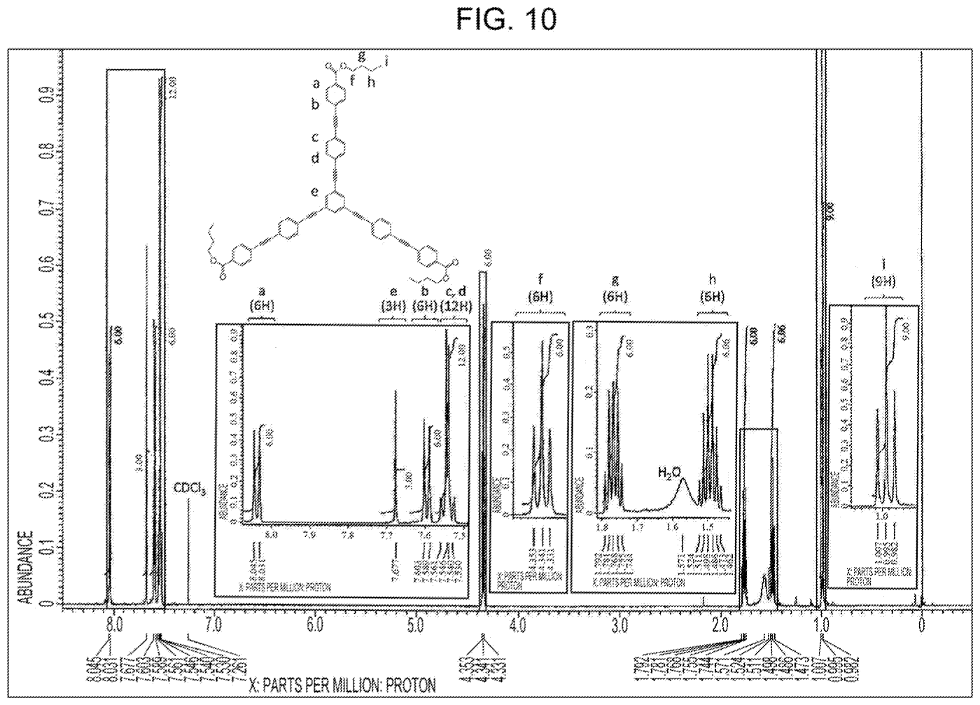

First, compound (8)-7 described above was added to a butanol solvent to form a suspension. Next, thionyl chloride was added to the suspension, and the mixture was heated under reflux overnight while performing stirring. A white solid was filtered off from the reaction solution and was washed with methanol. The solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (8)-9 as a white solid. Compound (8)-9 was identified by ¹H-NMR. FIG. 10 is a graph illustrating the ¹H-NMR spectrum of compound (8)-9. The ¹H-NMR spectrum of compound (8)-9 was as follows.

¹H-NMR (600 MHz, CHLOROFORM-D) δ 8.04 (d, J=8.3 Hz, 6H), 7.68 (s, 3H), 7.60 (d, J=8.3 Hz, 6H), 7.53-7.56 (m, 12H), 4.34 (t, J =6.5 Hz, 6H), 1.74-1.79 (m, 6H), 1.49 (td, J=15.0, 7.3 Hz, 6H), 0.99 (t, J=7.6 Hz, 9H).

### Synthesis of Compound (8)-10

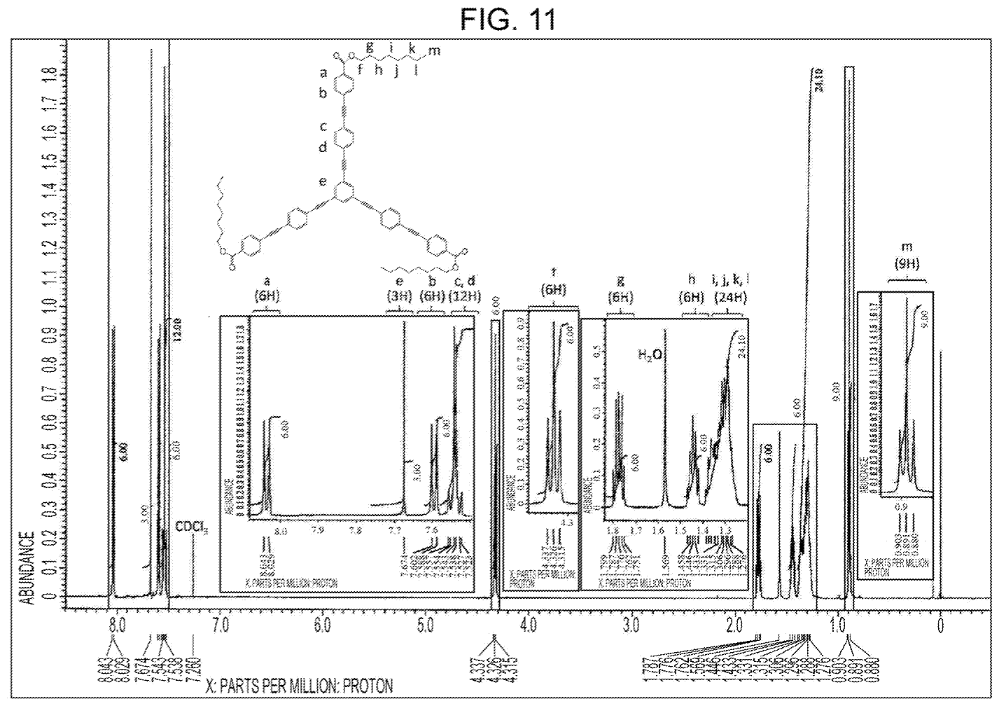

First, compound (8)-7 described above was added to an octanol solvent to form a suspension. Next, thionyl chloride was added to the suspension, and the mixture was heated under reflux overnight while performing stirring. A white solid was filtered off from the reaction solution and was washed with methanol. The solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (8)-10 as a white solid. Compound (8)-10 was identified by ¹H-NMR. FIG. 11 is a graph illustrating the ¹H-NMR spectrum of compound (8)-10. The ¹H-NMR spectrum of compound (8)-10 was as follows.

¹H-NMR (600 MHz, CHLOROFORM-D) δ 8.04 (d, J=8.3 Hz, 6H), 7.67 (s, 3H), 7.60 (d, J=8.3 Hz, 6H), 7.54 (td, J=9.1, 2.8 Hz, 12H), 4.33 (t, J=6.5 Hz, 6H), 1.75-1.80 (m, 6H), 1.42-1.47 (m, 6H), 1.27-1.38 (m, 24H), 0.89 (t, J=6.9 Hz, 9H).

### Synthesis of Compound (9)-7

First, precursor A of compound (8)-7 was prepared by the method described hereinabove. The precursor A and dimethyl 5-iodoisophthalate were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give a precursor of compound (9)-7.

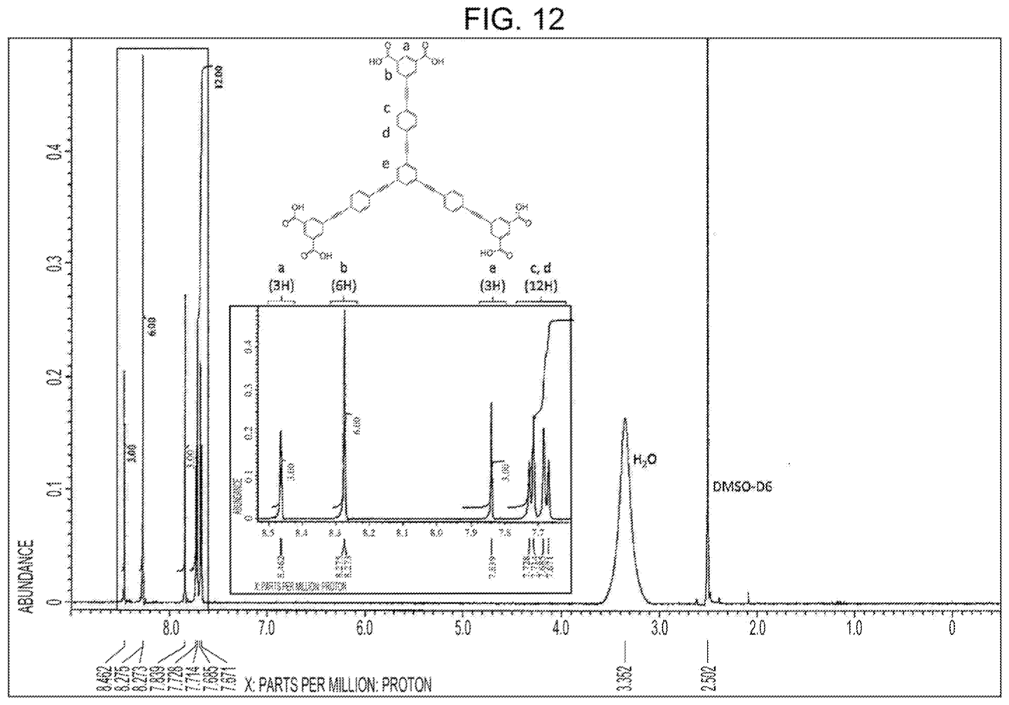

Next, the precursor of compound (9)-7 was dissolved into a mixed solution of tetrahydrofuran and methanol (v/v=1:1). An aqueous sodium hydroxide solution was added to the solution, and the mixture was heated under reflux overnight while performing stirring. After the reaction in the solution had completed, dilute hydrochloric acid was added to the solution. This addition acidified the solution and caused a solid to precipitate. The solid was washed with pure water to give compound (9)-7 as a white solid. Compound (9)-7 was identified by ¹H-NMR. FIG. 12 is a graph illustrating the ¹H-NMR spectrum of compound (9)-7. The ¹H-NMR spectrum of compound (9)-7 was as follows. ¹H-NMR (600 MHz, DMSO-D6) 6 8.46 (s, 3H), 8.27 (d, J=1.4 Hz, 6H), 7.84 (s, 3H), 7.72 (d, J=8.3 Hz, 6H) , 7.68 (d, J=8.3 Hz, 6H).

### Synthesis of Compound (10)-9

First, 1,3,5-tris[4′-(ethynyl)phenyl]benzene and 1-ethynyl-4-iodobenzene were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give precursor A of compound (10)-9.

Next, the precursor A of compound (10)-9 and methyl 4-iodobenzoate were dissolved into triethylamine. Catalytic amounts of triphenylphosphine, bis(triphenylphosphine)palladium (II) dichloride and copper (I) iodide were further added to the solution. Next, the solution was stirred at room temperature for 16 hours. Hydrochloric acid was added to the reaction solution for neutralization. Next, the reaction solution was extracted with ethyl acetate. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give precursor B of compound (10)-9.

Next, the precursor B of compound (10)-9 was dissolved into a mixed solution of tetrahydrofuran and methanol (v/v=1:1). An aqueous sodium hydroxide solution was added to the solution, and the mixture was heated under reflux overnight while performing stirring. After the reaction in the solution had completed, dilute hydrochloric acid was added to the solution. This addition acidified the solution and caused a solid to precipitate. The solid was washed with pure water to give precursor C (compound (10)-7)) of compound (10)-9 as a white solid.

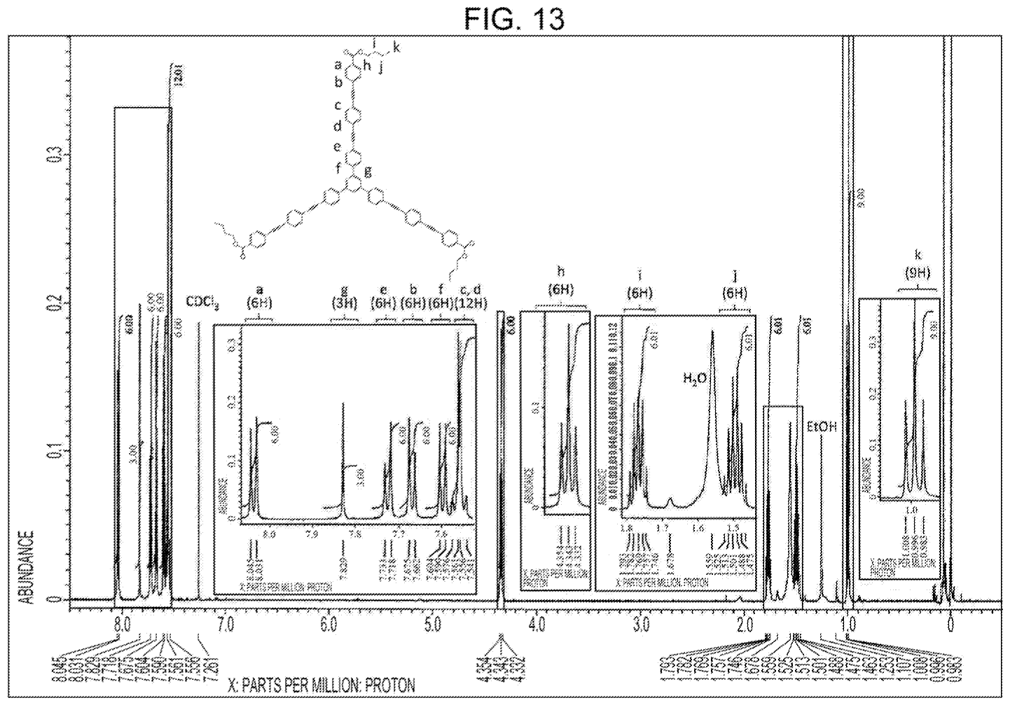

Next, the precursor C of compound (10)-9 was added to a butanol solvent to form a suspension. Next, thionyl chloride was added to the suspension, and the mixture was heated under reflux overnight while performing stirring. A white solid was filtered off from the reaction solution and was washed with methanol. The solid obtained was extracted with chloroform. Magnesium sulfate was added to the extract, and the extract was dehydrated. Next, the magnesium sulfate was filtered off from the extract. The filtrate thus obtained was concentrated using a rotary evaporator. The concentrate thus obtained was purified by silica gel column chromatography to give compound (10)-9 as a white solid. Compound (10)-9 was identified by ¹H-NMR. FIG. 13 is a graph illustrating the ¹H-NMR spectrum of compound (10)-9. The ¹H-NMR spectrum of compound (10)-9 was as follows.

¹H-NMR (600 MHz, CHLOROFORM-D) δ 8.04 (d, J=8.3 Hz, 6H), 7.83 (s, 3H), 7.73 (d, J =9.0 Hz, 6H), 7.67 (d, J=8.3 Hz, 6H), 7.60 (d, J=8.3 Hz, 6H), 7.56 (dd, J=11.7, 9.0 Hz, 12H), 4.34 (t, J=6.5 Hz, 6H), 1.75-1.79 (m, 6H), 1.49 (td, J=15.0, 7.3 Hz, 6H), 1.00 (t, J=7.6 Hz, 9H).

### Measurement of Two-photon Absorption Cross Section

The two-photon absorption cross section of the compounds synthesized was measured with respect to light having a wavelength of 405 nm. The measurement of two-photon absorption cross section was performed using the z-scan technique described in J. Opt. Soc. Am. B, 2003, Vol. 20, p. 529. The light source used for the measurement of two-photon absorption cross section was a Ti:sapphire pulsed laser. Specifically, a second high-frequency radiation of a Ti:sapphire pulsed laser was applied to the sample. The pulse width of the laser was 80 fs. The repetition frequency of the laser was 1 kHz. The average power of the laser was changed in the range of greater than or equal to 0.01 mW and less than or equal to 0.08 mW. The light from the laser had a wavelength of 405 nm. Specifically, the light from the laser had a central wavelength of greater than or equal to 402 nm and less than or equal to 404 nm. The full width at half maximum of the light from the laser was 4 nm.

### Prediction of Two-photon Absorption Cross Section

The two-photon absorption cross section was predicted of the synthesized compounds with respect to light having a wavelength of 405 nm. Specifically, the two-photon absorption cross section was calculated by the density functional theory (DFT) calculation based on the second-order non-linear response theory described in J. Chem. Theory Comput. 2018, Vol. 14, p. 807. Turbomole version 7.3.1 (manufactured by COSMOlogic) was used as software for the DFT calculation. def2-TZVP was used as the basis function. B3LYP was used as the functional.

The calculated value and the measured value of two-photon absorption cross section of the synthesized compounds were linearly regressed. In the linear regression, the coefficient of determination, R², was 0.9. This value confirmed high correlation between the calculated value and the measured value of two-photon absorption cross section. Next, the two-photon absorption cross section was calculated for other compounds differing from the synthesized compounds in the type of Z, using the regression equation obtained by the above linear regression.

### Measurement of Fluorescence Quantum Yield

The internal fluorescence quantum yield of the compounds synthesized was measured. A measurement sample was prepared by dissolving the compound into a dimethyl sulfoxide (DMSO) solvent. An absolute PL quantum yield measuring device (C9920-02 manufactured by Hamamatsu Photonics K.K.) was used for the measurement. The excitation wavelength was set to 325 nm. The measurement wavelengths were adjusted to the range of greater than or equal to 350 nm and less than or equal to 650 nm. A DMSO solvent was used as the reference.

### Measurement of Molar Absorption Coefficient

The compounds synthesized were analyzed by a method in accordance with JIS K0115: 2004 to measure the molar absorption coefficient. Specifically, first, an absorption spectrum of the measurement sample was measured. From the spectrum obtained, the absorbance at a wavelength of 405 nm was read. The molar absorption coefficient was calculated based on the concentration of the compound in the measurement sample and the optical path length of the cell used for the measurement.

### Prediction of Molar Absorption Coefficient

The molar absorption coefficient of the compounds synthesized was predicted. DFT calculation was used for the prediction of the molar absorption coefficient. Specifically, first, the excited-state calculation was performed with respect to the compound using quantum chemistry calculation program Gaussian 16 (manufactured by Gaussian). In the excited-state calculation, 6-31++G(d, p) was used as the basis function. CAM-B3LYP was used as the functional. By the excited-state calculation, the energy for exciting the compound and the probability of transition to the excited state were calculated. Further, from these calculation results, the absorption wavelengths and the oscillator strengths f at the respective absorption wavelengths were calculated. The oscillator strength is correlated with the molar absorption coefficient. Next, the half width was defined assuming a Gaussian absorption spectrum. Specifically, the half width was defined as 0.4 eV, and an absorption spectrum was drawn based on the absorption wavelengths and the oscillator strengths. The absorbance at a wavelength of 405 nm was read from the absorption spectrum thus obtained. This absorbance was taken as the calculated value of molar absorption coefficient.

Linear regression was performed on the calculated values and the measured values of molar absorption coefficient of compounds (12)-7, (13)-7, (8)-7, (9)-7 and (10)-9. In the linear regression, the coefficient of determination, R², was 0.9. This confirmed high correlation between the calculated value and the measured value of molar absorption coefficient. Next, the molar absorption coefficient was calculated for other compounds differing from the synthesized compounds in the type of Z, using the regression equation obtained by the above linear regression.

Tables 2 to 4 describe the measured and calculated values of two-photon absorption cross section, the fluorescence quantum yields, and the measured and calculated values of molar absorption coefficient determined as described hereinabove. In Tables 2 to 4, “No Data” means that no data was obtained.

TABLE 2

| | | | | Molar absorption | | |
|---|---|---|---|---|---|---|
| | | Two-photon absorption | | coefficient | | Fluorescence |
| | | cross section (GM) | | (L/(mol · cm)) | | quantum |
| | Compound | Measured | Calculated | Measured | Calculated | yield (%) |
| Ref. Ex. 1 | (12)-1 | 940 | 840 | 1 | 3 | 44 |
| Ex. 1 | (12)-2 | No Data | 850 | No Data | 2 | No Data |
| Ex. 2 | (12)-3 | No Data | 1000 | No Data | 4 | No Data |
| Ex. 3 | (12)-4 | No Data | 1050 | No Data | 4 | No Data |
| Ex. 4 | (12)-5 | No Data | 910 | No Data | 4 | No Data |
| Ex. 5 | (12)-6 | No Data | 880 | No Data | 4 | No Data |
| Ex. 6 | (12)-7 | 1300 | 1110 | 10 | 17 | 83 |
| Ex. 7 | (12)-8 | No Data | 1200 | No Data | 16 | No Data |
| Ex. 8 | (12)-9 | No Data | 1290 | 75 | 16 | 67 |
| Ex. 9 | (12)-10 | 1810 | 1330 | 75 | 16 | No Data |
| Ex. 10 | (12)-11 | No Data | 1110 | No Data | 23 | No Data |
| Ex. 11 | (12)-12 | No Data | 1100 | No Data | 10 | No Data |
| Ref. Ex. 2 | (12)-13 | No Data | 1180 | No Data | 15 | No Data |
| Ref. Ex. 3 | (12)-14 | No Data | 980 | No Data | 6 | No Data |
| Ex. 12 | (12)-15 | No Data | 1120 | No Data | 4 | No Data |
| Ex. 13 | (12)-16 | No Data | 1070 | No Data | 11 | No Data |
| Ex. 14 | (12)-17 | No Data | 1080 | No Data | 9 | No Data |
| Ex. 15 | (12)-18 | No Data | 1110 | No Data | 8 | No Data |

TABLE 3

| | | | | Molar absorption | | |
|---|---|---|---|---|---|---|
| | | Two-photon absorption | | coefficient | | Fluorescence |
| | | cross section (GM) | | (L/(mol · cm)) | | quantum |
| | Compound | Measured | Calculated | Measured | Calculated | yield (%) |
| Ex. 16 | (12)-19 | No Data | 1060 | No Data | 8 | No Data |
| Ex. 17 | (12)-20 | No Data | 1180 | No Data | 8 | No Data |
| Ex. 18 | (12)-21 | No Data | 950 | No Data | 15 | No Data |
| Ref. Ex. 4 | (12)-22 | No Data | 1120 | No Data | 35 | No Data |
| Ref. Ex. 5 | (12)-23 | No Data | 900 | No Data | 66 | No Data |
| Ex. 19 | (13)-7 | 700 | 530 | 20 | 3 | 36 |
| Ex. 20 | (13)-10 | 1200 | 1110 | 10 | 3 | 44 |
| Ex. 21 | (8)-1 | No Data | 1980 | No Data | 99 | No Data |
| Ex. 22 | (8)-2 | No Data | 1670 | No Data | 92 | No Data |
| Ex. 23 | (8)-3 | No Data | 2230 | No Data | 126 | No Data |
| Ex. 24 | (8)-4 | No Data | 2350 | No Data | 133 | No Data |
| Ex. 25 | (8)-5 | 2740 | 2100 | 185 | 126 | No Data |
| Ex. 26 | (8)-6 | No Data | 1670 | No Data | 137 | No Data |
| Ex. 27 | (8)-7 | 2080 | 2330 | 110 | 252 | 47 |
| Ex. 28 | (8)-8 | No Data | 2450 | No Data | 240 | No Data |
| Ex. 29 | (8)-9 | 2570 | 2640 | 110 | 245 | 79 |
| Ex. 30 | (8)-10 | 2720 | 3100 | 340 | 245 | No Data |

TABLE 4

| | | | | Molar absorption | | |
|---|---|---|---|---|---|---|
| | | Two-photon absorption | | coefficient | | Fluorescence |
| | | cross section (GM) | | (L/(mol · cm)) | | quantum |
| | Compound | Measured | Calculated | Measured | Calculated | yield (%) |
| Ex. 31 | (8)-11 | No Data | 2310 | No Data | 287 | No Data |
| Ex. 32 | (8)-12 | No Data | 2360 | No Data | 195 | No Data |
| Ex. 33 | (8)-13 | No Data | 2380 | No Data | 239 | No Data |
| Ex. 34 | (8)-14 | No Data | 2020 | No Data | 167 | No Data |
| Ex. 35 | (8)-15 | No Data | 2480 | No Data | 148 | No Data |
| Ex. 36 | (8)-16 | No Data | 2390 | No Data | 219 | No Data |
| Ex. 37 | (8)-17 | No Data | 2400 | No Data | 190 | No Data |
| Ex. 38 | (8)-18 | No Data | 2610 | No Data | 175 | No Data |
| Ex. 39 | (8)-19 | No Data | 2450 | No Data | 174 | No Data |
| Ex. 40 | (8)-20 | No Data | 2420 | No Data | 172 | No Data |
| Ex. 41 | (8)-21 | No Data | 1630 | No Data | 311 | No Data |
| Ex. 42 | (8)-22 | No Data | 1930 | No Data | 529 | No Data |
| Ex. 43 | (8)-23 | No Data | 1580 | No Data | 491 | No Data |
| Ex. 44 | (9)-7 | 1300 | 1080 | 70 | 102 | 35 |
| Ex. 45 | (10)-9 | 3310 | 3280 | 650 | 475 | 84 |

Next, compounds dissimilar to the compounds A of the formula (1) were provided. These compounds are described in Table 5 below. Compound if of Comparative Example 3 is represented by the following formula (16):

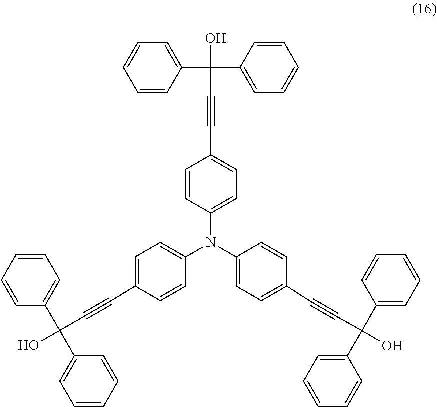 [SMILES: OC(C#Cc1ccc(N(c2ccc(C#CC(O)(c3ccccc3)c3ccccc3)cc2)c2ccc(C#CC(O)(c3ccccc3)c3ccccc3)cc2)cc1)(c1ccccc1)c1ccccc1]

Next, the compounds described in Table 5 were analyzed by the methods described hereinabove to measure the two-photon absorption cross section, the molar absorption coefficient and the fluorescence quantum yield. For 1,3,5-tris(4-carboxyphenyl)benzene of Comparative Example 1, the two-photon absorption cross section was also predicted. The results are described in Table 5. In Table 5, “No Data” means that no data was obtained.

TABLE 5

| | | | | Molar absorption | | |
|---|---|---|---|---|---|---|
| | | Two-photon absorption | | coefficient | | Fluorescence |
| | | cross section (GM) | | (L/(mol · cm)) | | quantum |
| | Compound | Measured | Calculated | Measured | Calculated | yield (%) |
| Comp. | 1,3,5-Tris(4-carboxyphenyl)benzene | 20 | 20 | 0 | No Data | 17 |
| Ex. 1 | (H₃BTB) | | | | | |
| Comp. | Hexakis(phenylethynyl)benzene (HPEB) | 23000 | No Data | 4010 | No Data | Greater than or |
| Ex. 2 | | | | | | equal to 10 |
| | | | | | | and less than |
| | | | | | | or equal to 30 |
| Comp. | 1f | 380 | No Data | 70 | No Data | 30 |
| Ex. 3 | | | | | | |
| Comp. | 4-Fluoro-4′-(phenylethynyl)benzophenone | 120 | No Data | 0 | No Data | No Data |
| Ex. 4 | | | | | | |
| Comp. | 1,1,3-Triphenyl-2-propyn-1-ol | 0 | No Data | 100 | No Data | No Data |
| Ex. 5 | | | | | | |
| Comp. | 1,4-Diphenylbutadiyne | 410 | No Data | 0 | No Data | No Data |
| Ex. 6 | | | | | | |
| Comp. | Benzo[h]quinoline | 10 | No Data | 0 | No Data | No Data |
| Ex. 7 | | | | | | |
| Comp. | 2-Isopropenylnaphthalene | 10 | No Data | 0 | No Data | No Data |
| Ex. 8 | | | | | | |

As can be seen from Tables 2 to 4, the compounds of Examples 1 to 45 corresponding to the compounds A of the formula (1) had a two-photon absorption cross section of greater than 500 GM when irradiated with light having a wavelength of 405 nm.

Further, the compounds of Examples 1 to 45 had a molar absorption coefficient of less than or equal to 650 L/(molcm) when irradiated with light having a wavelength of 405 nm. From these results, the compounds of Examples 1 to 45 were shown to have highly non-linear two-photon absorption characteristics with respect to light having a wavelength in the short wavelength region.

The hexasubstituted benzene used in Comparative Example 2, namely, hexakis(phenylethynyl)benzene had a large value of two-photon absorption cross section with respect to light having a wavelength of 405 nm, but its molar absorption coefficient was also high. Compared to a trisubstituted benzene, the hexasubstituted benzene has an extended π-electron conjugated system and thus the single-photon absorption peak tends to shift to a longer wavelength region. In the compound of Comparative Example 2, part of the wavelengths giving rise to a single-photon absorption peak probably overlapped with the wavelength of the excitation light, and consequently the molar absorption coefficient was increased. Compounds A represented by the formula (1) are trisubstituted benzenes and have an extended π-electron conjugated system. The highly non-linear two-photon absorption characteristics of the compounds A probably stem from this structure.

Further, the compounds of Examples 6, 8, 19, 27, 29, 44 and 45 each had a fluorescence quantum yield of greater than or equal to 35%. This shows that the compounds A represented by the formula (1) tend to have a high fluorescence quantum yield.

The light-absorbing materials of the present disclosure may be used in applications such as, for example, recording layers in three-dimensional optical memories, and photocurable resin compositions for stereolithography. The light-absorbing materials of the present disclosure have highly non-linear two-photon absorption characteristics with respect to light having a wavelength in the short wavelength region. Thus, the light-absorbing materials of the present disclosure can realize very high spatial resolution in applications such as three-dimensional optical memories and modeling machines. Further, the light-absorbing materials of the present disclosure tend to have a high fluorescence quantum yield. Thus, the light-absorbing material, when used in a recording layer of a three-dimensional optical memory, allows for reading of the ON/OFF state of the recording layer based on a change in fluorescence from the light-absorbing material. The light-absorbing materials of the present disclosure may also be used as fluorescent dye materials in, for example, two-photon fluorescence microscopes.

What is claimed is:

1. A light-absorbing material comprising a compound represented by the following formula (1): 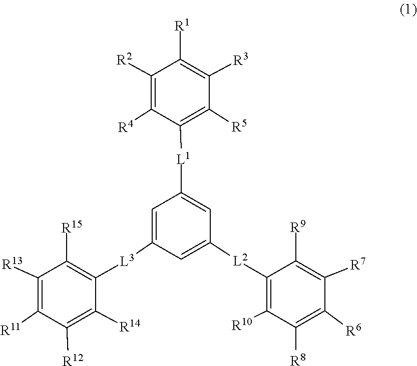
wherein R¹ to R¹⁵ each independently include at least one atom selected from the group consisting of H, C, N, O, F, P, S, Cl, I and Br, and L¹ to L³ are each independently represented by the following formula (2) or (3): 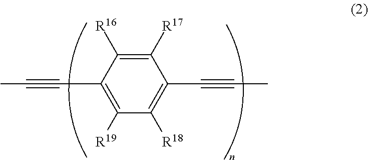
wherein R¹⁶ to R¹⁹ each independently include at least one atom selected from the group consisting of H, C, N, O, F, P, S, Cl, I and Br, and n is an integer of 1 to 3, 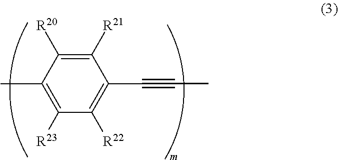
wherein R²⁰ to R²³ each independently include at least one atom selected from the group consisting of H, C, N, O, F, P, S, Cl, I and Br, and m is an integer of 1 to 3,
when the compound is represented by the formula (4) below, at least one selected from the group consisting of R¹, R⁶ and R¹¹ is a halogen atom, an alkyl group, an alkyl halide group, an unsaturated hydrocarbon group, a hydroxyl group, a carboxyl group, an alkoxycarbonyl group, an acyl group, an amide group, an acyloxy group, a thiol group, an alkylthio group, a sulfonic acid group, an acylthio group, an alkylsulfonyl group, a sulfonamide group, a primary amino group or a secondary amino group,

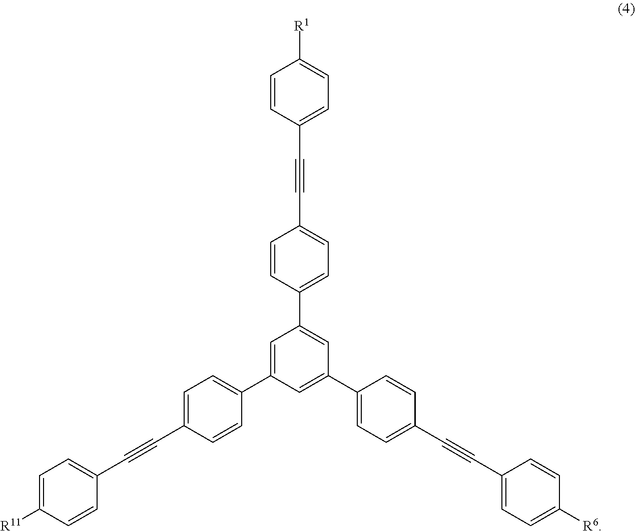

2. The light-absorbing material according to claim 1, wherein
when the compound is represented by the formula (5) below, at least one selected from the group consisting of R¹, R⁶ and R¹¹ is a halogen atom, an alkyl group having two or more carbon atoms, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a primary amino group or a secondary amino group,

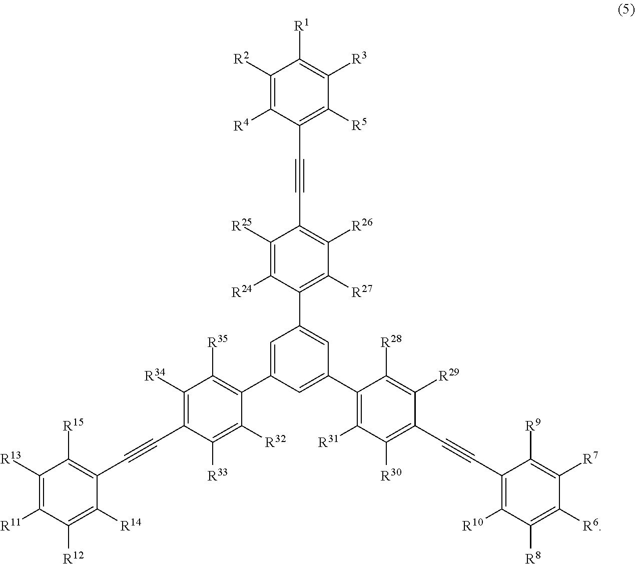

3. The light-absorbing material according to claim 1, wherein
when the compound is represented by the formula (17) below, at least one selected from the group consisting of R¹, R⁶ and R¹¹ is a halogen atom, an alkyl group having two or more carbon atoms, an alkyl halide group, a vinyl group, a hydroxyl group, a carboxyl group, an alkoxycarbonyl group, an acyl group, an amide group, an acyloxy group, an alkylthio group, a sulfonic acid group, an acylthio group, an alkylsulfonyl group, a sulfonamide group, a silyl group, a primary amino group or a secondary amino group,

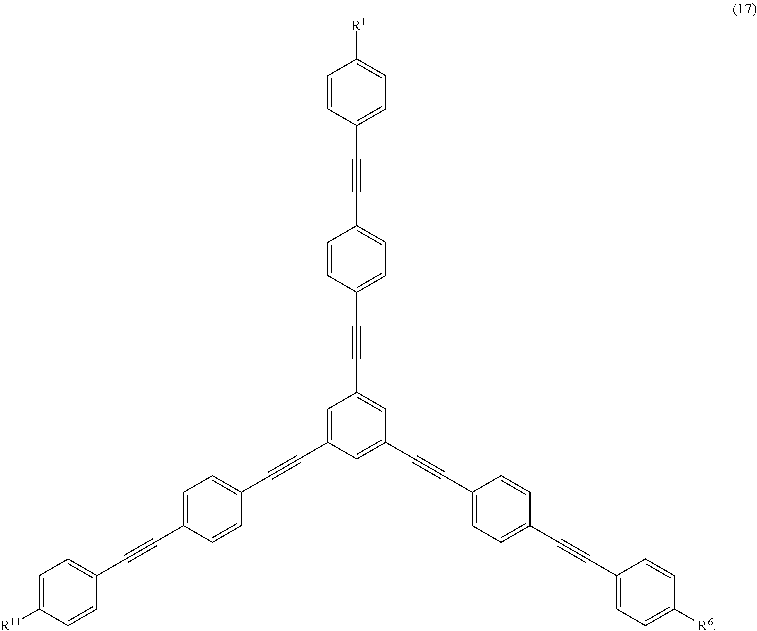

4. The light-absorbing material according to claim 1, wherein L¹ to L³ in the compound are each represented by the formula (2).

5. The light-absorbing material according to claim 4, wherein the compound is represented by the following formula (5): 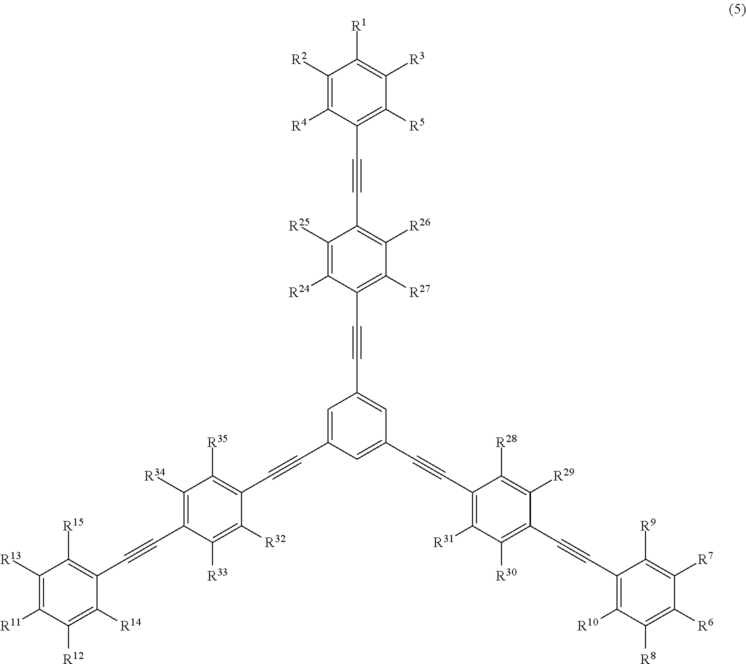
wherein R²⁴ to R³⁵ each independently include at least one atom selected from the group consisting of H, C, N, O, F, P, S, Cl, I and Br.

6. The light-absorbing material according to claim 1, wherein
L¹ to L³ in the compound are each represented by the formula (3).

7. The light-absorbing material according to claim 6, wherein
the compound is represented by the following formula (6): 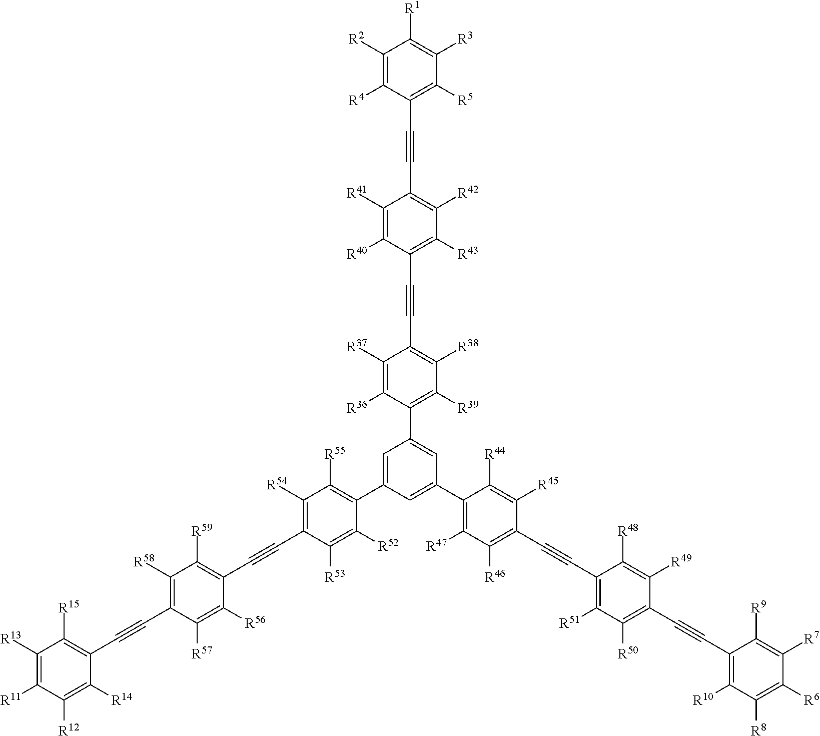
wherein R³⁶ to R⁵⁹ each independently include at least one atom selected from the group consisting of H, C, N, O, F, P, S, Cl, I and Br.

8. The light-absorbing material according to claim 6, wherein
the compound is represented by the following formula (7): 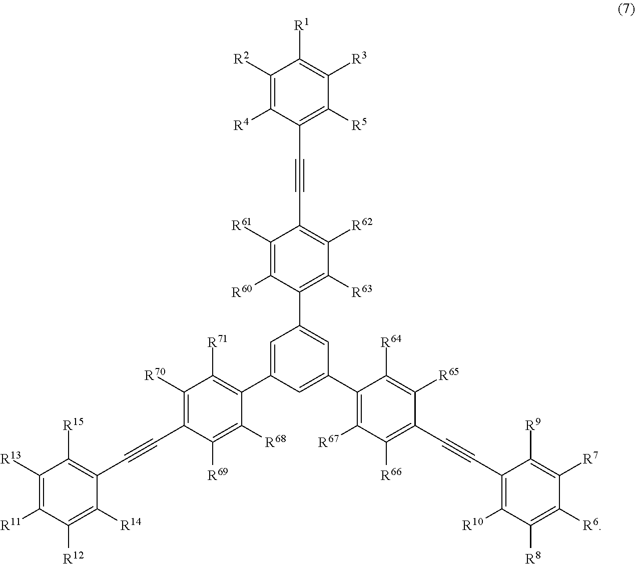
wherein R⁶⁰ to R⁷¹ each independently include at least one atom selected from the group consisting of H, C, N, O, F, P, S, Cl, I and Br.

9. The light-absorbing material according to claim 1, wherein
R¹ to R¹⁵ are each independently a hydrogen atom, a halogen atom, an alkyl group, an alkyl halide group, an unsaturated hydrocarbon group, a hydroxyl group, a carboxyl group, an alkoxycarbonyl group, an acyl group, an amide group, a nitrile group, an alkoxy group, an acyloxy group, a thiol group, an alkylthio group, a sulfonic acid group, an acylthio group, an alkylsulfonyl group, a sulfonamide group, a primary amino group, a secondary amino group, a tertiary amino group or a nitro group.

10. The light-absorbing material according to claim 1, wherein
at least one selected from the group consisting of R¹ to R³, R⁶ to R⁸ and R¹¹ to R¹³ is an electron-donating group or an electron-withdrawing group.

11. The light-absorbing material according to claim 10, wherein
the electron-withdrawing group is a carboxyl group or an alkoxycarbonyl group.

12. The light-absorbing material according to claim 10, wherein
the electron-withdrawing group is —COO(CH₂)₃CH₃ or —COO(CH₂)₇CH₃.

13. A light-absorbing material used in a device utilizing light having a wavelength of greater than or equal to 390 nm and less than or equal to 420 nm,
the light-absorbing material comprising a compound represented by the following formula (1): 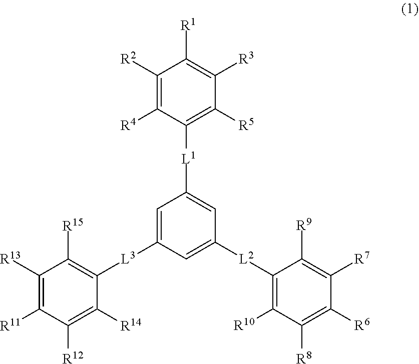
wherein R¹ to R¹⁵ each independently include at least one atom selected from the group consisting of H, C, N, O, F, P, S, Cl, I and Br, and L¹ to L³ are each independently represented by the following formula (2) or (3): 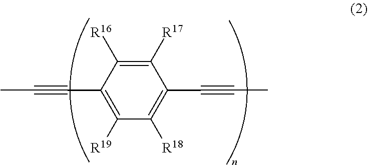
wherein R¹⁶ to R¹⁹ each independently include at least one atom selected from the group consisting of H, C, N, O, F, P, S, Cl, I and Br, and n is an integer of 1 to 3, 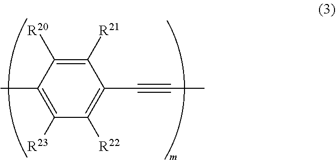
wherein R²⁰ to R²³ each independently include at least one atom selected from the group consisting of H, C, N, O, F, P, S, Cl, I and Br, and m is an integer of 1 to 3.

14. A recording medium comprising a recording film containing a light-absorbing material containing a compound represented by the following formula (1): 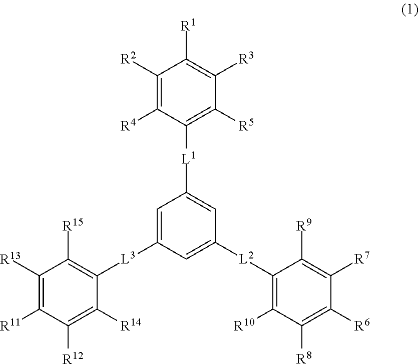
wherein R¹ to R¹⁵ each independently include at least one atom selected from the group consisting of H, C, N, O, F, P, S, Cl, I and Br, and L¹ to L³ are each independently represented by the following formula (2) or (3): 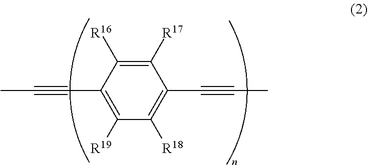
wherein R¹⁶ to R¹⁹ each independently include at least one atom selected from the group consisting of H, C, N, O, F, P, S, Cl, I and Br, and n is an integer of 1 to 3, 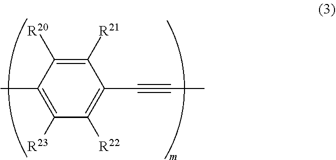
wherein R²⁰ to R²³ each independently include at least one atom selected from the group consisting of H, C, N, O, F, P, S, Cl, I and Br, and m is an integer of 1 to 3.

15. An information recording method comprising:
providing a light source that emits light having a wavelength of greater than or equal to 390 nm and less than or equal to 420 nm; and
focusing the light from the light source with a lens and applying the light to a recording region in a recording medium including a recording film containing a light- absorbing material containing a compound represented by the following formula (1): 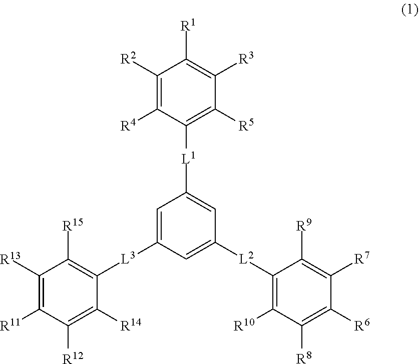
wherein R¹ to R¹⁵ each independently include at least one atom selected from the group consisting of H, C, N, O, F, P, S, Cl, I and Br, and L¹ to L³ are each independently represented by the following formula (2) or (3): 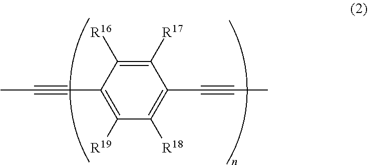
wherein R¹⁶ to R¹⁹ each independently include at least one atom selected from the group consisting of H, C, N, O, F, P, S, Cl, I and Br, and n is an integer of 1 to 3, 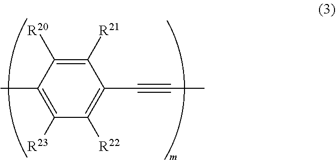
wherein R²⁰ to R²³ each independently include at least one atom selected from the group consisting of H, C, N, O, F, P, S, Cl, I and Br, and m is an integer of 1 to 3.

16. An information reading method for reading information recorded by the information recording method according to claim 15, the information reading method comprising:
applying light to the recording region in the recording medium and measuring an optical characteristic of the recording region; and
determining whether or not information is recorded in the recording region based on the optical characteristic.

17. The information reading method according to claim 16, wherein the optical characteristic is an intensity of fluorescence emitted from the recording region.
